(19) United States
(12) Patent Application Publication
(10) Pub. No.: US 2019/0073403 A1
(43) Pub. Date: Mar. 7, 2019

(54) QUERYING DATA USING MASTER TERMINOLOGY DATA MODEL

(71) Applicant: TriNetX, Inc., Cambridge, MA (US)

(72) Inventors: David Fusari, Groton, MA (US); Matvey B. Palchuk, Acton, MA (US); Asad Saad Basir, Boston, MA (US); Joshua Owen Graff, Somerville, MA (US); Steve Kundrot, Belmont, MA (US); Merryl J. Gross, Watertown, MA (US)

(21) Appl. No.: 16/084,836

(22) PCT Filed: Mar. 13, 2017
(86) PCT No.: PCT/US17/22124

§ 371 Date: Sep. 13, 2018

Related U.S. Application Data
(60) Provisional application No. 62/307,961, filed on Mar. 14, 2016.

(51) Int. Cl. G06F 17/30; G16H 50/70

(52) U.S. Cl. CPC G06F 17/30554; G06F 17/30566; G06F 17/30522; G16H 50/70

(57) ABSTRACT

A method, a system, and a computer program product for querying data are disclosed. A query to a database is received. The data in the database is arranged using a master terminology data model. The master terminology data model contains a mapping of one or more terminology structures. Data responsive to the query is generated.

### CROSS-REFERENCE TO RELATED APPLICATIONS

The present application claims priority to U.S. Provisional Patent Application No. 62/307,961 to Fusari et al., filed Mar. 14, 2016, and entitled “Querying Data Using Master Terminology Data Model,” and incorporates its disclosure herein by reference in its entirety.

The present application relates to International Patent Application No. PCT/US2014/069369, filed Dec. 9, 2014, which claims priority to U.S. Provisional Patent Appl. No. 61/913,809 to Fusari, filed Dec. 9, 2013, and incorporates their disclosures herein by reference in their entireties.

### TECHNICAL FIELD

In some implementations, the current subject matter relates to data processing and in particular, to querying data using a master terminology.

### BACKGROUND

Clinical trials focused on oncology typically require information about cancer that is not captured in billing diagnoses like ICD-9. Specifically, most frequently required information is (1) primary tumor site (organ location of the primary tumor, such as breast, lung, etc.); (2) characteristics of the tumor, including the type of tumor cells (i.e., histology), the tumor cell behavior (degree of invasiveness of the tumor), and the tumor grade (degree of cell differentiation); and (3) staging—severity of disease, characterized by tumor size, lymph node involvement and presence of metastasis. This information is frequently required to adequately describe an oncologic disease. In today's world, genetic biomarkers are increasing in importance in oncology as more knowledge is gained about cancer genomics and more targeted cancer therapies are developed. Unlike billing diagnoses (ICD-9), oncology information is typically not captured in a structured fashion in a typical electronic medical record (“EMR”). However, cancer is a reportable disease, and every provider is required to report cancer cases to a state cancer registry. There are standards in place for gathering information required for this reporting. The data is captured in a structured fashion and is typically stored in databases referred to as cancer or tumor registries.

### SUMMARY

In some implementations, the current subject matter relates to a computer-implemented method for querying data. The method can include receiving a query to a database, where the data in the database can be arranged using a master terminology data model, wherein the master terminology data model can contain a mapping of one or more terminology structures, and generating data responsive to the query.

In some implementations, the structured master terminology data model can use a mapping of terms in two or more terminology structures, e.g., ICD-10 and ICD-O. The structured data model can be a new type of terminology structure (e.g., cancer terminology structure), where the structure can include a plurality of levels (level 0: “Tumor Registry” (e.g., top level), level 1: tumor site (or any other aspect of the cancer, such as, for example, but not limited to, biomarker(s), mutation(s), genomic biomarker(s), etc., and/or any combination thereof), etc.). Data can be mapped and structured using various aspects of the oncology data (e.g., tumor site, morphology (histology and behavior), tumor grade, tumor stage, cancer-specific factors, treatment, recurrence, multiple primary diagnoses, etc.). Further, specific data can be mapped between existing terminology structures using specific aspects of the cancer (e.g., diagnoses, sites, biomarkers, mutations, etc.) to provide additional oncology data in the master terminology for assisting user in building/running of queries. In some implementations, synonyms in the oncology terminology can be used for the purposes of creating the master terminology data model. In some implementations, a provider map to represent oncology data (e.g., tumor morphology, site-to-morphology, oncology qualifiers, etc.) can be generated so that the data can be appropriately loaded in accordance with the master terminology for querying purposes. In some implementations, the queries can be generated in free form/text and then translated into appropriate parameters based on the master terminology, where the resulting data can be presented via a user interface and/or in any other fashion. The queries can also be built using specific codes of the master terminology.

In some implementations, the current subject matter relates to a computer-implemented method for querying data. The method can include receiving a query to a database, obtaining, based on at least one parameter of the query, data from the database responsive to the query by traversing the database in accordance with the mapping, and providing the data responsive to the query in accordance with the at least one of: the at least one determined site element and the at least one determined referenced element. The data can be stored in accordance with at least one data model. The data model can contain at least one data node storing data and can be structured in accordance with at least one master terminology containing a mapping of a plurality of terminology structures. The parameter can be an element of a first terminology structure in the plurality of terminology structures. The traversal can include at least one of the following: determining, based on the at least one parameter, at least one site element contained in a second terminology structure in the plurality of terminology structures, where the site element can identify data in the database for inclusion in the data responsive to the query, and determine, based on the parameter, at least one referenced element contained in the second terminology structure, where the referenced element can identify data in the database being related to the data responsive to the query.

In some implementations, the current subject matter can include one or more of the following optional features. The first terminology structure can include terminology from International Classification of Disease (ICD-10) and the second terminology structure can include terminology from International Classification of Disease—Oncology (ICD-O). At least one site element can identify at least one of the following: a site of a tumor in a body of a patient, a tumor type, a biomarker, a mutation, a genomic biomarker, a genomic biomarker mutation, and any combination thereof. At least one referenced element can be determined based on the at least one site element. At least one referenced element can include at least one of the following: a tumor stage, a tumor grade, at least one cancer specific factor, at least one treatment, a tumor recurrence, at least one multiple primary diagnosis, morphology, and any combination thereof. Morphology can be determined based on the second terminology structure.

In some implementations, data can be obtained by selecting, based on the morphology, data responsive to the query.

In some implementations, at least one referenced element can include at least one of the following: a tumor stage, a tumor grade, at least one cancer specific factor, at least one treatment, a tumor recurrence, at least one multiple primary diagnosis, and any combination thereof. At least one site element can contain a morphology determined based on the parameter using the first terminology structure. Data in the database corresponding to the morphology can be included in the data responsive to the query.

In some implementations, the current subject matter can implement a tangibly embodied machine-readable medium embodying instructions that, when performed, cause one or more machines (e.g., computers, etc.) to result in operations described herein. Similarly, computer systems are also described that can include a processor and a memory coupled to the processor. The memory can include one or more programs that cause the processor to perform one or more of the operations described herein. Additionally, computer systems may include additional specialized processing units that are able to apply a single instruction to multiple data points in parallel. Such units include but are not limited to so-called “Graphics Processing Units (GPU).”

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE FIGURES

The accompanying drawings, which are incorporated in and constitute a part of this specification, show certain aspects of the subject matter disclosed herein and, together with the description, help explain some of the principles associated with the disclosed implementations. In the drawings,

FIG. 1 illustrates an exemplary system for identifying candidates for clinical trials, according to some implementation of the current subject matter;

FIG. 2 illustrates an exemplary method, according to some implementation of the current subject matter;

FIG. 3 illustrates an exemplary system architecture for performing identification of patient candidates for clinical trials, according to some implementations of the current subject matter;

FIG. 4 illustrates an exemplary tumor registry chart that contains information cancer specific parameters (i.e., “primary site”, “morphology”, “date of diagnosis”, “stage”, “TNM”, “grade”, “cancer-specific factors”, and “treatment”).

FIG. 5 illustrates additional details chart with regard to the “treatment” factor shown in FIG. 4.

FIG. 6 illustrates an exemplary modeling process, which can be used to organize primary top-level site to organize individual observations from the tumor registry (as shown in FIGS. 4-5).

FIG. 7 illustrates an exemplary site-specific oncology data model, according to some implementations;

FIG. 8 illustrates an exemplary non-site-specific oncology data model, according to some implementations;

FIG. 9 illustrates an exemplary Hodgkin's disease table;

FIGS. 10a-n illustrate exemplary interfaces containing mappings associated with various queries, according to some implementations of the current subject matter;

FIG. 11 illustrates an exemplary system, according to some implementations of the current subject matter; and

FIG. 12 illustrates an exemplary method, according to some implementations of the current subject matter.

### DETAILED DESCRIPTION

In some implementations, the current subject matter relates to a method and a system for processing data, and in particular, to querying data using a master terminology data model. Data to be queried can be arranged using such master terminology, which can be a data model containing mapping(s) and/or cross-mapping(s) of terms from various terminology structures (e.g., ICD-9, ICD-10 and ICD-O, and/or any other terminology structures and/or standards). Data can be loaded and/or stored in a database using the master terminology. The database can be associated with a data owner, user, and/or provider. For example, in a medical field, a healthcare provider (e.g., a hospital, a medical clinic, a doctor's office, a laboratory, a network of medical service providers, etc., and/or any combination thereof.

Various users can query the stored data using free-from text, terms associated with the master terminology, structured query language, etc., and/or any combination thereof. The queries can be based on, but are not limited to, inclusion/exclusion criteria, demographic data, medical conditions, timing, etc. The queries can be entered via a user interface that may be communicatively coupled (e.g., via a network, such as the Internet, intranet, extranet, metropolitan area network (“MAN”), wide area network (“WAN”), local area network (“LAN”), virtual local area network (“VLAN”), wireless networks, wired networks, etc., and/or any other networks and/or any combination thereof) to the location of where the data has been uploaded and/or stored. As a result of executing queries, a search of a database(s) in the provider network can be conducted. The search can be performed locally and/or over a network. Execution of the query can be performed on a single database and/or across one or more databases (e.g., a network of databases). The databases in a network of database can be communicatively coupled using one or more networks described above.

The search can allow accessing and searching de-identified patient data, identified patient data, and/or any other type of data, and/or any combination thereof. The search can generate result(s), including various statistical analyses, where the results from various network sites and/or databases can be aggregated and provided to the user. An exemplary way to search data is disclosed in co-owned, co-pending U.S. patent application Ser. No. 15/102,848 to Fusari et al., filed Jun. 8, 2016, which claims priority to International Patent Application No. PCT/US2014/069369, filed Dec. 9, 2014, which claims priority to U.S. Provisional Patent Appl. No. 61/913,809 to Fusari et al., filed Dec. 9, 2013, the disclosures of which are incorporated herein by reference in their entireties.

In some implementations, the current subject matter system can be, but is not limited to, implemented in any industry, including pharmaceutical industry, medical industry, research (e.g., medical, scientific, etc.) research industry, telecommunications industry, academia, etc. The following describes exemplary implementations of the current subject matter system as applicable to identification of potential cancer patients and/or their conditions along with various specifics. Such identification can be used for the purposes of conducting clinical trial(s), a clinical study, clinical research, outcomes research, population health and monitoring, quality of care, etc. (e.g., for a drug, a medical device, etc.), as for example disclosed in co-owned, co-pending U.S. patent application Ser. No. 15/102,848 to Fusari et al., filed Jun. 8, 2016, which claims priority to International Patent Application No. PCT/US2014/069369, filed Dec. 9, 2014, which claims priority to U.S. Provisional Patent Appl. No. 61/913,809 to Fusari et al., filed Dec. 9, 2013, the disclosures of which are incorporated herein by reference in their entireties.

The following discussion relates to querying data that has been loaded and/or stored based on a data model developed using a mapping of ICD-9, ICD-10 and ICD-O terminology structures and/or terminology standards. The mapping can be a master terminology that can be used for querying the data. ICD-O is a domain-specific extension of the International Statistical Classification of Diseases and Related Health Problems (“ICD”) for tumor diseases. ICD-10 contains codes for diseases, signs and symptoms, abnormal findings, complaints, social circumstances, and external causes of injury or diseases, and includes a list of morphology codes contained in the ICD-O. The queried data can be a federated data that can be located behind a firewall of a data provider (e.g., hospital, a clinic, a medical facility, and/or any other facility) and can be appropriately de-identified, if necessary. As a result of a query, a list of cancer subjects and/or cancer specific conditions can be generated for the purposes of, for example, conducting a clinical study, a clinical trial, clinical research, outcomes research, population health and monitoring, quality of care, etc., and/or any other purposes. As can be understood, the current subject matter is not limited to the above exemplary implementation and other uses of the subject matter's processes are possible. For ease of illustration, the following discussion will refer to clinical trials.

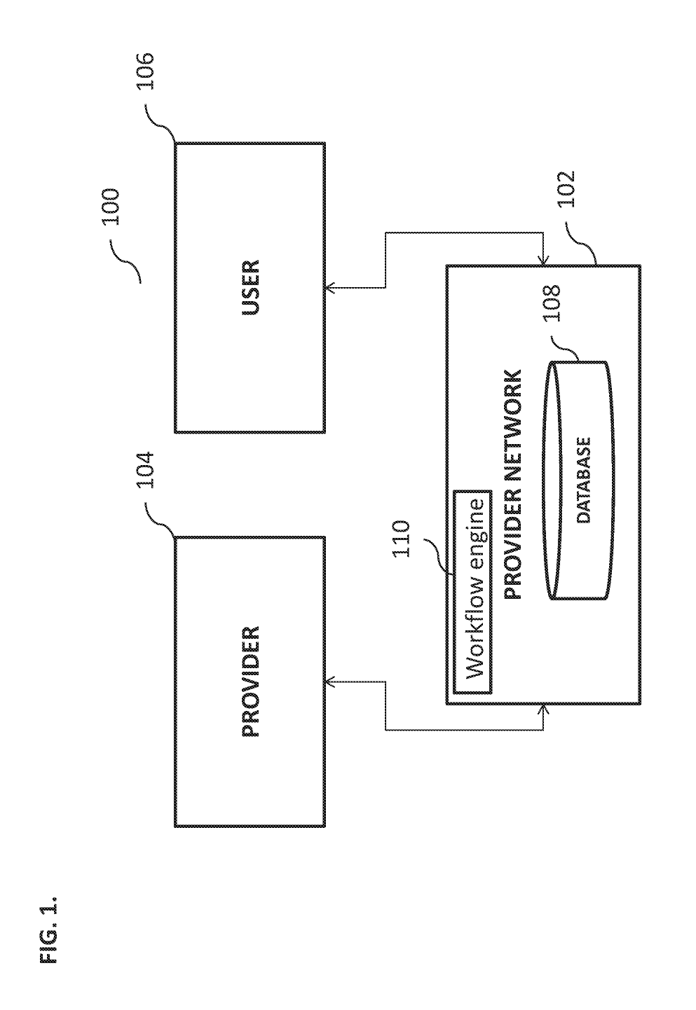

FIG. 1 illustrates an exemplary system 100 for querying data using a master terminology (e.g., for the purposes of identifying candidates for clinical trials), according to some implementations of the current subject matter. An exemplary system 100 is disclosed in co-owned, co-pending U.S. patent application Ser. No. 15/102,848 to Fusari et al., filed Jun. 8, 2016, which claims priority to International Patent Application No. PCT/US2014/069369, filed Dec. 9, 2014, which claims priority to U.S. Provisional Patent Appl. No. 61/913,809 to Fusari et al., filed Dec. 9, 2013, the disclosures of which are incorporated herein by reference in their entireties.

The system 100 can include a provider network 102 that can include one or more databases 108 and a workflow engine 110, one or more providers 104 and one or more users 106. The providers 104 can be hospitals, clinics, governmental agencies, private institutions, academic institutions, medical professionals, public companies, private companies, and/or any other individuals and/or entities and/or any combination thereof. The provider network 102 can be a network of computing devices, servers, databases, etc., which can be connected to one another via using various network communication capabilities (e.g., Internet, local area network (“LAN”), metropolitan area network (“MAN”), wide area network (“WAN”), and/or any other network, including wired and/or wireless). Some or all entities in the network 102 can have various processing capabilities that can allow users of the network 102 to query and obtain data related to the patients, where the data can be stored in one or more databases 108. The database 108 can include requisite hardware and/or software to store various data related to patients, where the data can be de-identified. The data can also contain various statistical counts of patients derived from the de-identified data.

The users 106 can be researchers and/or any other users, including but not limited to, hospitals, clinics, governmental agencies, private institutions, academic institutions, medical professionals, public companies, private companies, and/or any other individuals and/or entities and/or any combination thereof. In some implementations, the user(s) 106 can be a single individual and/or multiple individuals (and/or computing systems, software applications, business process applications, business objects, etc.). The user(s) 106 can be separate from the provider 104, such as being a part of a pharmaceutical company, and/or can be part of the provider 104 (e.g., an individual at a hospital, a research institution, etc.).

In non-limiting, exemplary implementations, users 106 can be designing protocols for the study and/or analysis and/or research. The study can involve a new study, an existing study, and/or any combination thereof. It can be based on existing data, data to be obtained, projected data, expected data, a hypothesis, and/or any other data. The users 106 can query the data contained in one or more databases 108, where the query can relate to an identification of candidates for clinical trial(s) or for any other purpose. The queries can be written in and/or translated to any known computer language. The queries can be entered into a user interface displayed on a user's computer terminal.

In some implementations, the data, e.g., patient data, can be stored locally in one or more databases of the data providers. Alternatively, the data can be stored at a remote database and/or a network of databases. The query can be executed on one database at a time and/or on some or all databases simultaneously. The databases in a network can be associated with different providers.

In some implementations, the current subject matter can allow users and/or providers and/or any other third parties to generate a query in one language, format, etc., translate the query to the language, format, etc. of the location that contains the requested data, and generate an output to the issuer of the query. This can allow for a smooth interaction between users 106 and/or providers 104, i.e., the providers do not need to perform any kind of translation of user's queries into their own language, format, etc. In some implementations, the system 100 can be configured to store information about provider's data and how it is stored (e.g., location, language, format, structure, etc.) and how it should be queried. In some implementations, providers and/or users can submit to the system 100 their requirements and/or preferences as to how they wish queries of data should be submitted. This information can be provided manually and/or automatically by the users/providers. In some implementations, the system 100 can also contain a dictionary of terms that can be used to translate queries from one system (e.g., user system) to another (e.g., provider system) and vice versa. The dictionary can assist in resolving various discrepancies between terms that may be used by the users and/or providers. The above functionalities can be integrated into the network 102 and/or be part of the workflow engine 110. In some implementations, the results of the search (which can be related to that data, and is de-identified) can be stored centrally.

The system 100 and its network provider 102 can further include a workflow engine and/or a computing platform 110 that can be used to coordinate activities between providers and/or between pharmaceutical company and providers. The workflow engine 110 can be a computing interface (e.g., an application programming interface) and/or any other computing mechanism that can receive, format, execute, transmit, etc. queries as well as receive, format, etc. results of queries. The workflow engine 110 can coordinate data requests, queries, data analysis, and/or output to ensure that the data requests are processed efficiently. For example, when a researcher at pharmaceutical company wants to initiate a chart review, the workflow engine 110 can manage coordination of the request to one or more data providers that can be performing the chart review, coordinating the responses, and returning the results back to the requester. In some exemplary implementations, connecting a researcher to a provider can also require multiple approvals within the provider organization before the researcher can execute the chart review.

The system 100 can be designed, for example, to allow clinical researchers at different organizations the ability to mine through significant amounts of clinical records and patient history for a number of different purposes. Researchers at pharmaceutical companies can use the system to improve clinical trial designs avoiding the possibility of having to amend the trial and losing valuable time and money in the effort to bring clinical trials to market. Hospital researchers can collaborate with other selected hospitals that are also part of the network 102 on certain diseases and treatment efficacy across a broad population of patients. Hospitals and providers can also use the system to search their own patient database. As can be understood, other users can also use the system to obtain requisite information.

The current subject matter system 100 can integrate a network of provider organizations where patient data never leaves the providers data center. Queries can be federated across providers in real time and only aggregated counts and other statistical characteristics of the results based on the query are returned to the user. A simple example can be a query for all people diagnosed with diabetes between the ages of 40 and 50. What is returned can be a count of the people that have that diagnosis and are between the ages of 40 and 50. A set of other statistics can be also returned (e.g., how many are male and how many are female, a more fine grained age breakdown, counts of the different medications patients are on, etc.).

The system 100 can be delivered as a web application to end users and can be cloud hosted. The system can be hosted on cloud-hosted services and can include software that can be deployed behind the data provider firewalls. In some implementations, a secured and/or private network can be implemented, whereby access to the network and/or data contained therein can be restricted to members of the network. In some implementations, no special software and/or hardware and/or any combination thereof may be required behind a providers firewall. In some implementations, data providers can be hospitals, academic institutions, governmental agencies, public and/or private companies, clinics, medical providers, third party aggregators of clinical data, and/or any other individuals and/or entities.

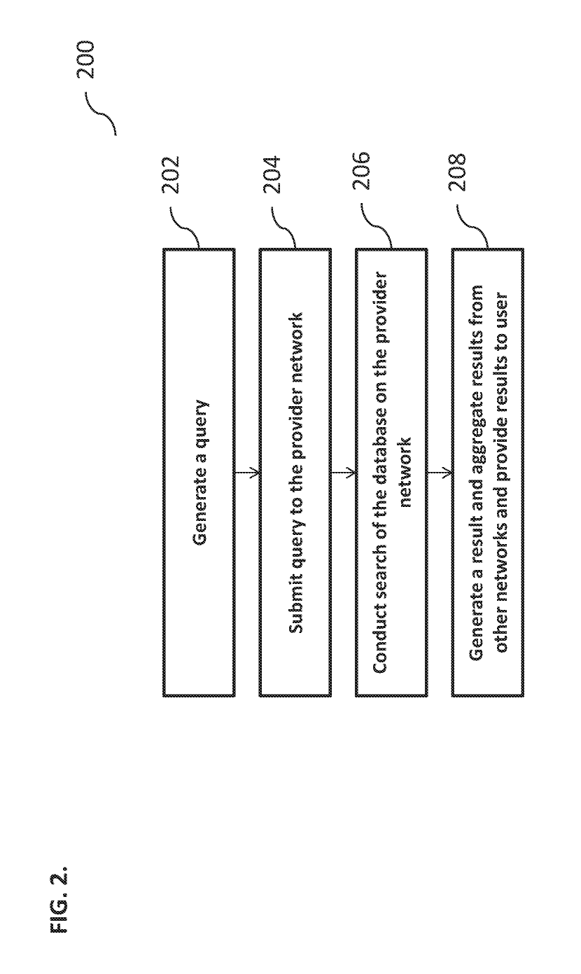

FIG. 2 illustrates an exemplary method 200, according to some implementations of the current subject matter. An exemplary process 200 is disclosed in co-owned, co-pending U.S. patent application Ser. No. 15/102,848 to Fusari et al., filed Jun. 8, 2016, which claims priority to International Patent Application No. PCT/US2014/069369, filed Dec. 9, 2014, which claims priority to U.S. Provisional Patent Appl. No. 61/913,809 to Fusari et al., filed Dec. 9, 2013, the disclosures of which are incorporated herein by reference in their entireties. At 202, user 106 can generate queries based on clinical study objectives and/or assumptions and/or other parameters. The query can be submitted to the network 102, at 204. The queries can be based on, but are not limited to, inclusion/exclusion criteria, demographic data, aspects of the disease, etc. A search of the database(s) 108 can be conducted, at 206. The search can be performed locally or over a network of databases and can search de-identified patient data. The search can generate a result, including various statistical analyses, at 208, where the results from various network sites and/or databases can be aggregated and provided to the user 106.

In some implementations, users can execute queries on data that can be stored on various selected network sites. This can allow users to collaborate on patient recruitment feasibility, trial design, and/or site selection.

In some implementations, some exemplary users 106 can include, but are not limited to, individuals and/or entities at biotech and pharmaceutical organizations that can make use of the resulting data for research and workflow coordination with healthcare organizations in support of clinical trial design and execution. In some implementations, biotech and/or pharmaceutical company users can never have access to de-identified or identified patient data, and they can only have access to statistical information (counts) about a patient population across providers.

In some implementations, some exemplary users 106 can include, but are not limited to, researchers/investigators at provider organizations that are interested in initiating their own research, or collaborating with company users in a workflow activity. These users can have access to de-identified and/or identified patient data depending on the nature of the policies enforced by the individual provider. As can be understood, other users and/or groups of users can have various access rights to the data. In some implementations, specific users can be granted access to particular data but can be excluded from accessing other data that may be stored in a database.

In some implementations, the current subject matter can also support exploratory research, which can allow users to ascertain population of patient candidates, including various attributes of the patients in the population (e.g., medical conditions, age, location, relationship to the provider, etc.). For example, when considering a study for cancer patients, a study physician can identify a cohort of patients with a cancer diagnosis, and then explore a range of medications, laboratories, co-morbidities, procedures, and/or any other characteristics of the cohort.

In some implementations, data responsive to the query can be represented in a user-friendly and intuitive way. The data can be encoded, such as, by using standard clinical coding schemes like ICD-9, ICD-10, ICD-O, and/or any other type of coding for diagnosis, LOINC codes for lab tests and results, CPT codes for procedures, and RxNorm (or in some cases SNOMED) for medications. As can be understood, any other ways of coding the data responsive to the query can be used. Users performing a query do not need to know the specific codes, although if they are known, they can be used to find the correct term. In some implementations, the current subject matter can include an auto-complete feature that can allow the user to begin typing any term and the system can list similar terms based on heuristic matching logic to speed the use of the system and make it simple to specify the requisite criteria. For each term, the user can see how many patients have that specific diagnosis, lab, procedure, medication prescription, etc. across the entire network of millions of accessible de-identified patient records.

In some implementations, queries performed by the user and/or their results can be stored and identified as being related to the study that the user desires to conduct. The information can be stored in a database and/or any other memory location. The queries and corresponding results can be compared based on various parameters, e.g., identified patients, medical conditions, locations, etc. In some implementations, the results of the queries and/or the studies can be shared with third parties and can be used to track various activities relating to the studies.

In some implementations, the current subject matter can provide at least one of the following functionalities: query building, result reporting, provider collaboration, data quality and ontology tools, administration tools, development infrastructure, preparatory chart review, site identification/selection, peer review, patient recruitment, as well as other functions.

In some implementations, the query building functionality can include at least one of the following: auto completion of query terms, providing a number of patients that match each query term, applying parameters to query terms when applicable, specifying a date range for any query term, applying Boolean logic to the query terms, automatic tracking of query history, and/or any other functionalities, as will be discussed in further detail below. The results reporting functionality can include at least one of the following, providing a number of patients matching the query criteria, providing age and gender breakdown, providing patient counts by provider, providing patient diagnosis/comorbidities, providing patient laboratory results and/or values, listing patient medications and/or procedures, and/or any other functionalities, as will be discussed in further detail below. The provider collaboration functionality can include at least one of the following: creation of a network of providers, constraining search criteria to a field of study, tracking activity of providers, grouping membership workflow processes, and/or any other functionalities. The data quality and ontology tools can include at least one of the following: tools to develop and/or manage master ontology, mappings to master ontology, providing information about anomalies and/or inconsistencies, testing query harness for on-boarding provider to verify performance, etc. The administrative tools can include at least one of the following: provider and user management, provider setup and configuration, system monitoring, infrastructure notifications upon occurrence of application and/or system errors, audit log access and/or review, etc. The development infrastructure functionalities can include at least one of the following: development tools and infrastructure, defect tracking, development and test environments, automated build and regression testing, source code management, etc.

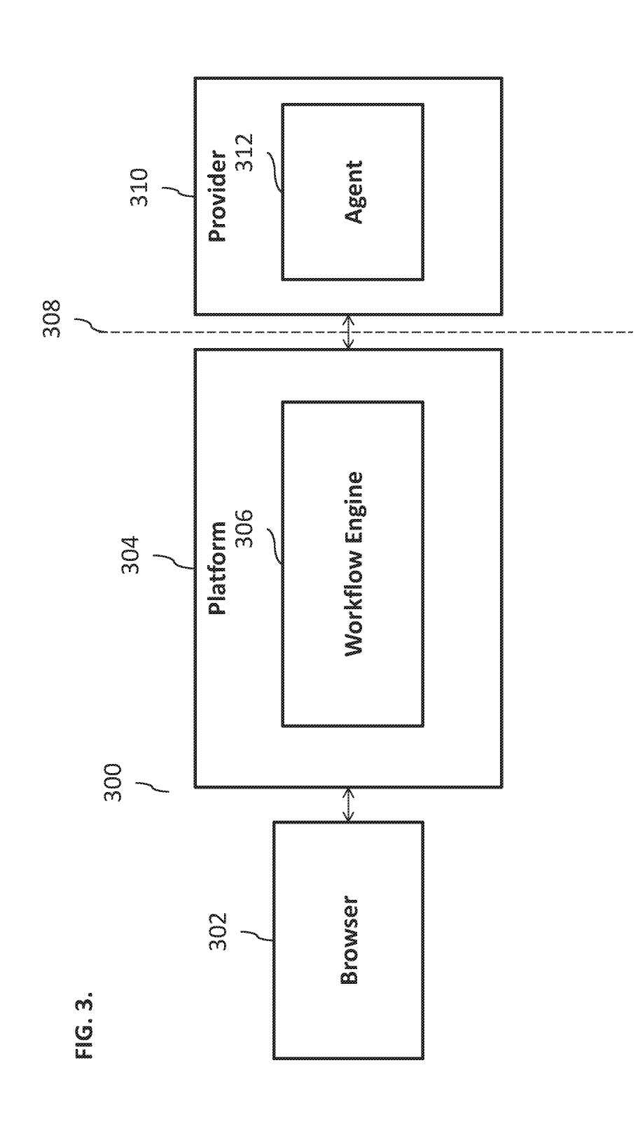

FIG. 3 illustrates an exemplary system architecture 300 for querying data stored in a database in accordance with a data model (e.g., generated as result of a mapping of two or more registries (e.g., ICD-10 and ICD-O)), according to some implementations of the current subject matter. The system can include a browser component 302, a platform component 304 that can include a workflow engine 306, a firewall component 308, and a provider component 310. The browser component 302 can be used by the user 106 (as shown in FIG. 1) to generate queries, access various data, and/or perform any other functionalities. The platform component 304 can be software, hardware, and/or any combination thereof and can be included in the provider network component 102 (as shown in FIG. 1), where the workflow engine 306 can be similar to the workflow engine 110 (as shown in FIG. 1). The platform can be a software-as-a-service (“SaaS”) platform where entities using the platform can manage their own users, their own access controls, and/or control their own configuration. The provider 310 can include a platform agent 312 that can provide access for the provider to the platform 304 and the user 302 and vice versa. The agent 312 can be software, hardware, and/or any combination thereof. In some implementations, the agent 312 can be installed on the provider system. Alternatively, the agent 312 is not used and the provider can directly access the platform 304.

The firewall 308 can provide appropriate security to the data being exchanged between the provider 310, the user 302, and the platform 304. In some implementations, to enhance security of the data being exchanged and/or accessed by the platform 304, the agent 312 installed on the provider system can communicate with the platform 304 without requiring any listening communication ports to be open. In some implementations, any patient data, identified and/or de-identified, may never leave the provider's data center and/or control unless specific authorization to access that information is received and/or granted. All access to patient data and/or platform 304 can require secure authentication and all activity can be audited.

In some implementations, the platform 304 can be a combination of an enterprise application and a cloud hosted multi-tenant SaaS application. The cloud-hosted SaaS infrastructure can provide core management and/or administration services, web application for clinical research, and/or can manage workflow activities for coordination of various workflow activities. In some implementations, the platform 304 can also include a database (e.g., database 108 shown in FIG. 1) that can be a cloud-hosted instance of a relational database. This database can store queries, query results, user identities, configuration information, master ontology, data mappings, metadata, etc. This database can be automatically replicated and backed up for high availability.

In some implementations, the current subject matter can allow a user to query and/or navigate through oncology specific terminology and/or all of the related concepts in an intuitive way. The querying/navigation can be performed for solid and/or fluid based tumors and/or any other cancers (and/or any other types of diseases). Using the current subject matter system, the user can also gain understanding of clinical characteristics of oncology patients. The current subject matter can be implemented using informatics for integrating biology and the bedside (“i2b2”), which can be a tool for organizing and analyzing clinical data. The data that the user can query can be delivered to providers and loaded using an i2b2 oncology ontology.

The oncology data is typically organized using specific parameters, such as site, morphology (histology and behavior), grade, staging, cancer-specific factors, treatment, recurrence, multiple primary diagnoses, etc. Each of these parameters is discussed below.

### Site

World Health Organization has a standard called International Classification of Disease—Oncology (ICD-O). ICD-O has coded descriptions of tumor sites or topologies (see, e.g., http://codes.iarc.fr/topography). There are 70 top-level primary disease sites such as breast, colon, prostate, etc. The codes begin with letter C and are followed by two-digit number (e.g., colon is C18). Each top-level site is subdivided into sub-sites. For example, colon is subdivided into ascending, transverse and descending colon segments. Those are coded with letter C followed by two-digit number followed by a period and one more digit (e.g., C18.1, C18.2, etc.).

### Morphology

The same ICD-O standard has descriptions of tumor tissue and behavior. The tumor tissue type, or histology, describes the kind of cells that comprise the tumor. ICD-0 has 174 major histologies, such as adenocarcinoma, sarcoma, neuroblastoma, etc. These are represented by a three-digit numeric code from 800 to 999. Each major histology is subdivided into more specific histologies, represented by a four-digit code. For example, adenocarcinoma (e.g., 814) is subdivided into such histologies as scirrhous adenocarcinoma (e.g., 8141), monomorphic adenoma (e.g., 8146), basal cell adenocarcinoma (e.g., 8147), etc.

Tumor behavior characterizes the degree of invasiveness of the tumor. There are various types of tumor behavior, each represented by a single-digit numeric code, such as by of a non-limiting example:
0: Benign neoplasms
1: Neoplasms of uncertain and unknown behavior
2: In situ neoplasms
3: Malignant neoplasms stated or presumed to be primary
6: Malignant neoplasms, stated or presumed to be secondary

ICD-O combines histology and behavior into a single code, referred to as morphology (see, e.g., http://codes.iarc.fr/codegroup/2), together known as tumor morphology. A morphology code is a four-digit histology code followed by a behavior code separated by a forward slash. For example, 8500/2 is ductal carcinoma in situ (“DCIS”)—a common type of breast cancer.

At each body site, cancers can arise with specific kinds of morphologies; morphologies differ by site. For each top-level site, there is an associated list of morphology codes that are applicable to this site.

### Grade

In addition to morphology, another useful description of tumors is their grade, defined as degree to which cells lose their differentiation. The list of grades is provided by ICD-O and is fixed at these values:
1: Low grade—Well-differentiated
2: Intermediate grade—Moderately differentiated
3: High grade—Poorly differentiated

### Staging

Tumor staging is used to describe overall severity of the disease. Stages vary by cancer site, but there is an overall similarity: Stage 0 is typically a small and non-invasive tumor (carcinoma in situ), Stages I, II, and III describe more extensive disease as tumor size increases and it invades surrounding tissues, and Stage IV represents cancer that spread to distant tissues or organs, or metastasized. Stage is determined by a system known as TNM. TNM is a combination of three variables: tumor size (“T”), lymph nodes involved (“N”), and presence of metastasis (“M”). TNM is the predominant staging system in use today. Two organizations—the Union for International Cancer Control (“UICC”) and the American Joint Committee on Cancer (“AJCC”)—are behind the development of cancer staging systems. The organizations agreed to unify their efforts into a single system in 1987. Note that tumor staging is not represented by ICD-O standard.

### Cancer-Specific Factors

Tumor registries collect additional cancer-specific information. These data are modeled as entity/value pairs in North American Association of Central Cancer Registries (“NAACCR”). Each cancer has a variable number of these “factors” or questions and a pre-defined vocabulary for answers (typically enumerated lists of answers). The data collected in specific factors is of crucial importance for individual cancers. Unfortunately, there is no direct mapping between ICD-O top-level sites and NAACCR cancer-specific facts, necessitating linking them manually.

### Treatment

The following top level treatment modalities are available:
Chemotherapy
Diagnostic (ex, biopsy)
Endocrine Treatment
Hormone therapy
Immunotherapy
Other treatment
Palliative
Radiation
Surgery
Transplant Procedure

Some of these have child nodes. For example, “Chemotherapy, multiple agents (combination regimen)” and “Chemotherapy, single agent” are found under Chemotherapy. The sequence of treatments may also be noted (such as chemotherapy or radiation given before and/or after surgery). This treatment information can be specified in clinical trials eligibility criteria, as patients must be either treatment naive (no prior treatment) or refractory (not responsive to prior treatment). While the treatment may also be obtained from the ICD-9 procedure data, it may be more directly available from the tumor registry data.

### Recurrence

Recurrence documents first recurrence of the tumor either locally, regionally or at a distant site. There is also a modifier “Months from initial Dx to 1st Recurrence” with values in months.

### Multiple Primary Diagnoses

The following facts are available regarding multiple primaries:
Multiple malignant primaries
Multiple non-malignant primaries
Single malignant primary only (no multiple)
Single non-malignant primary only (no multiple)

Typically, users looking for oncology data search for top-level sites and those will act as the “concepts” in the query builder; all other (or majority of) oncology data will be selected based on that top-level concept. In some implementations, the current subject matter can allow users to search for data that might not be based on a particular oncological diagnosis. The users can enter any search term, which can correspond to any level and/or any type of information (e.g., site, diagnosis, treatment, biomarker, genomic biomarker, genomic biomarker mutation, tumor biomarker, etc., which may or not be tied and/or mapped to ICD-10/ICD-O) and obtain relevant data (e.g., subjects having a similar biomarker, etc.). In some implementations, the current subject matter can allow providers (e.g., hospitals, clinics, etc.) can load their data in accordance with the current subject matter's defined schema. The schema can be developed based on term mappings that can deliver a model where the user does not have to traverse through multiple coding systems to assemble a meaningful query.

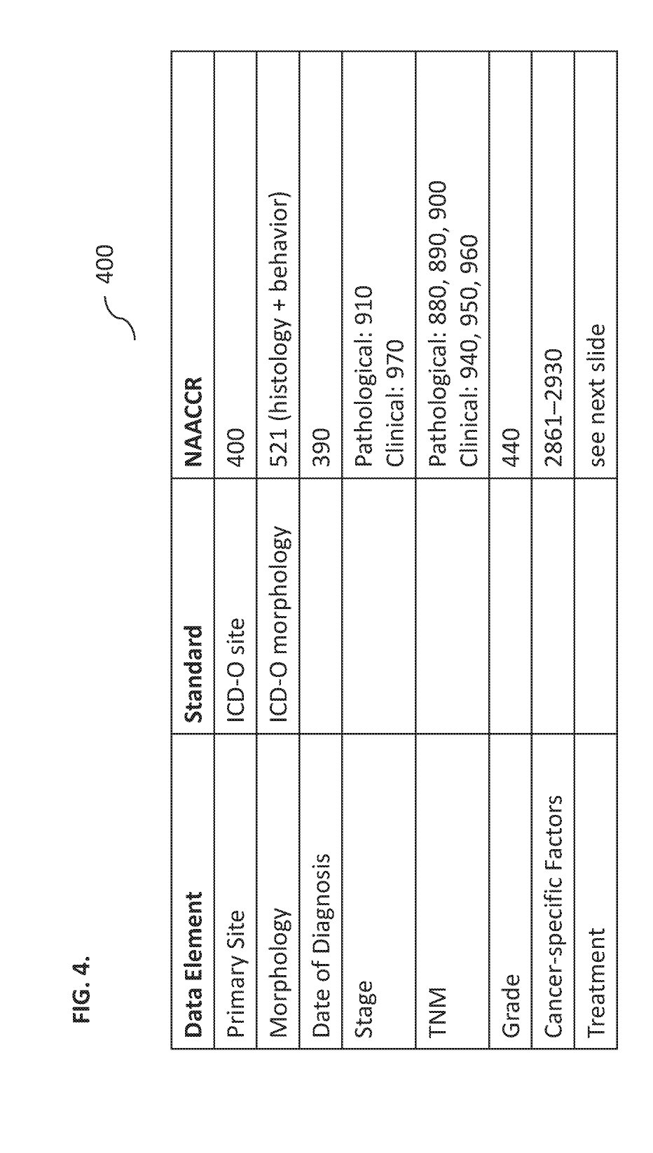

FIG. 4 illustrates an exemplary tumor registry chart 400 that contains information cancer specific parameters (i.e., “primary site”, “morphology”, “date of diagnosis”, “stage”, “TNM”, “grade”, “cancer-specific factors”, and “treatment”). As shown in FIG. 4, the exemplary cancer has a primary site identified as ICD-O site and an NAACCR value of 400. Its morphology parameter is ICD-O morphology having a value of 521, which represents histology and behavior of the cancer. The stage parameter of the cancer (as diagnosed on a specific data) has a pathological NAACCR value of 910 and clinical value of 970. The TNM parameter also identifies pathological NAACCR values (e.g., 880,890, 900), and clinical NAACCR values (e.g., 940, 950, 960). The grade and cancer specific factors parameters also include corresponding values (e.g., 440 and 2861-2930, respectively). Each of these parameters illustrates various characteristics of the cancer that may have been diagnosed on a specific date.

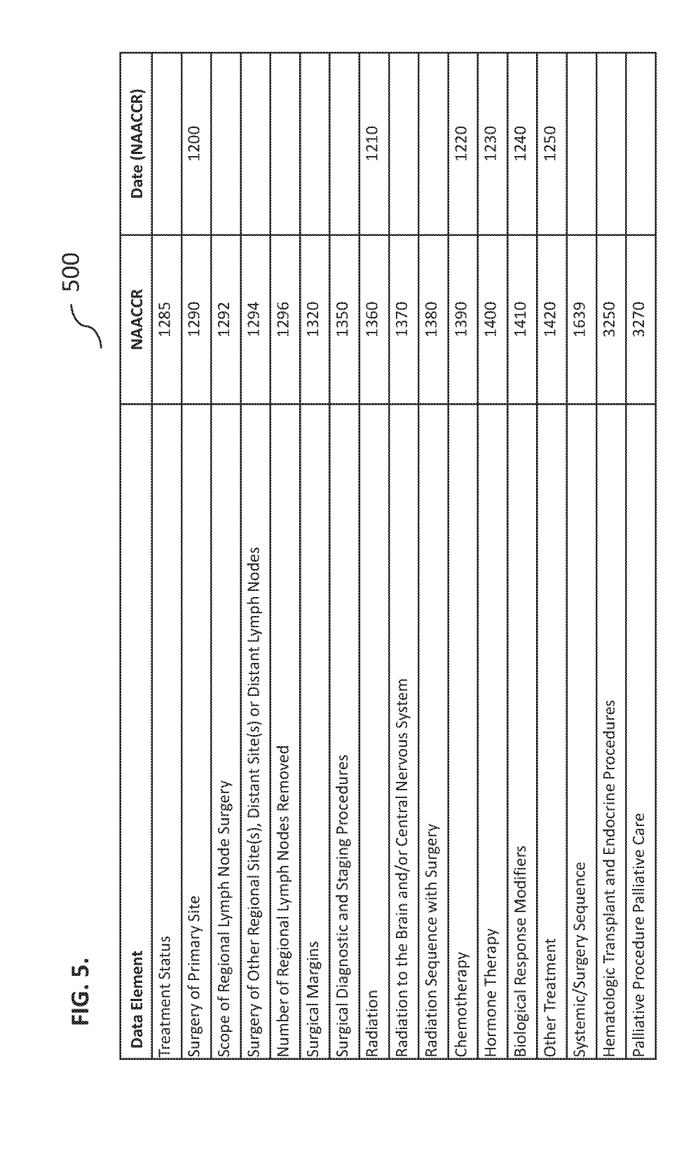

FIG. 5 is an exemplary chart 500 that shows additional details chart 400 with respect to the “treatment” parameter shown in FIG. 4. The details can include “treatment status”, “surgery of primary site”, etc., as shown in FIG. 5. Each of the parameters shown in FIG. 5 also has corresponding NAACCR value and NAACCR date value. For example, the “treatment status” parameter can have a NAACCR value of 1285 and the “surgery of primary site” can have a NAACCR value of 1290 with a date value 1200. As shown in FIGS. 4-5, each factor can be associated with a specific NAACCR code and standard. An exemplary tumor terminology structure analysis is shown in Appendix A.

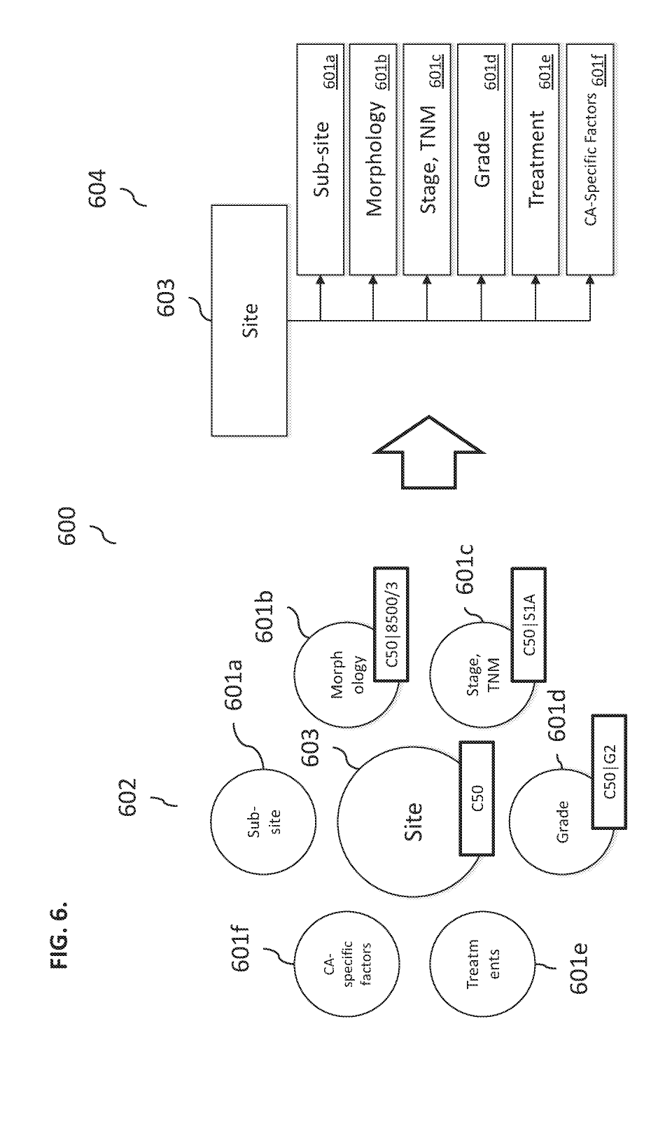

FIG. 6 illustrates an exemplary modeling process 600, which can be used to organize primary top-level site and individual observations from the tumor terminology structure (as shown in FIGS. 4-5), according to some implementations of the current subject matter. As shown in FIG. 6, the model can include a structure 602 (e.g., a tumor terminology structure) that can further include one or more levels or nodes 603 and 601 (a, b c, d, e, f) (in the following description the words level and node are used interchangeably). The node 603 can be a center node or a root node of the structure 602 and nodes 601 can be related to and/or dependent on the node 603. The tumor terminology structure 602 can include a primary site (e.g., C50) node 603 for a particular cancer. The primary site node 603 can include a sub-site node 601a, morphology (e.g., C50|8500/3) node 601b, stage and TNM (e.g., C50|S1A) node 601c, a grade (e.g., C50|G2) node 601d, treatment(s) node 601e, and CA specific factors node 601f. The current subject matter can be used to restructure or organize the tumor terminology structure 602 into a hierarchical representation data model 604, where each site node 603 can be a root node and can be associated with sub-site(s), morphology(ies), stage(s)/TNM, grade(s), CA-specific factor(s), and treatment(s) nodes 601.

Once the data is organized in the hierarchical representation data model 604, the data model 604 can be provided to data providers (e.g., hospitals, clinics, etc.) for the purposes of having their data loaded into their databases (e.g., federated databases) in accordance with the provided data model. The provider databases and/or other types of storage structures can be arranged using the data model 604. Any existing and/or new information regarding cancer cases (and/or any other diseases) can be converted and stored using the data model 604.

In some implementations, once the data has been uploaded into the providers' database in accordance with the provided data model 604, users can search for and find cancers of interest (such as, using ICD-10-CM diagnoses terminology). In some implementations, the terminology can be enriched using synonyms. ICD-9-CM can be interleaved into the terminology and/or customized based on general equivalence mappings (“GEMs”), which can be a mapping tool that can perform a crosswalk between, for example, ICD-9 and ICD-10.

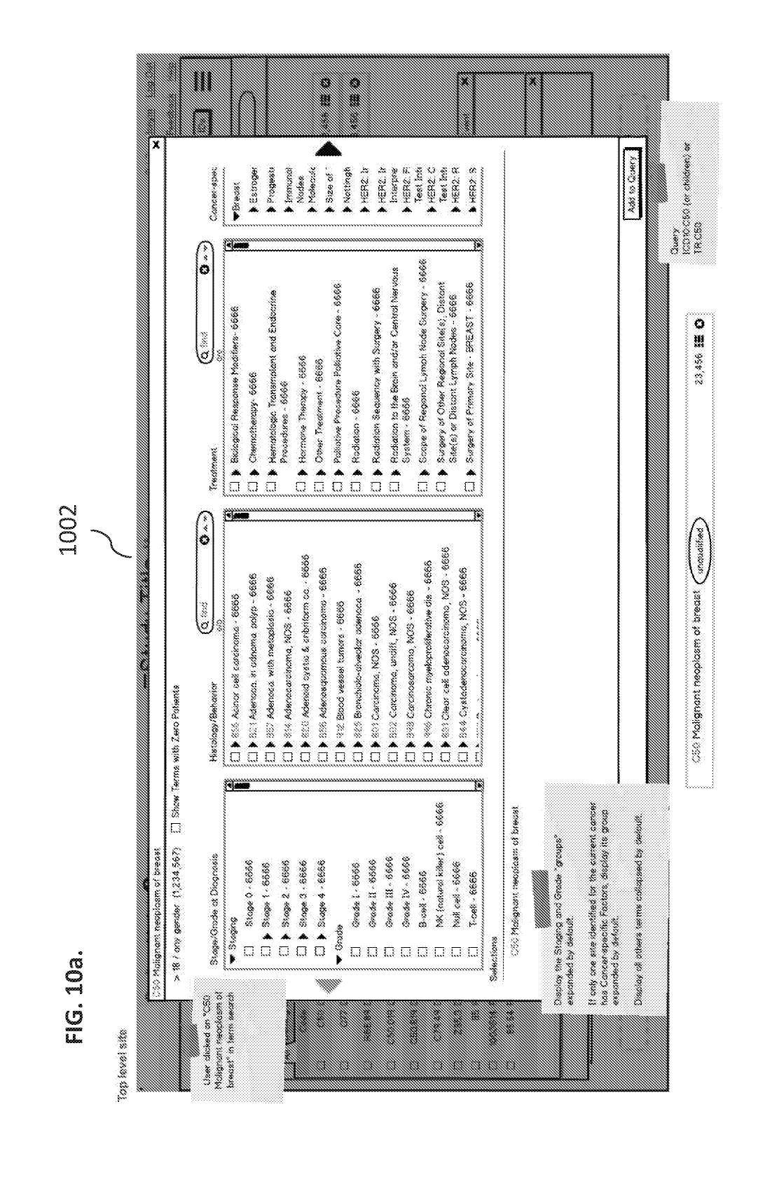

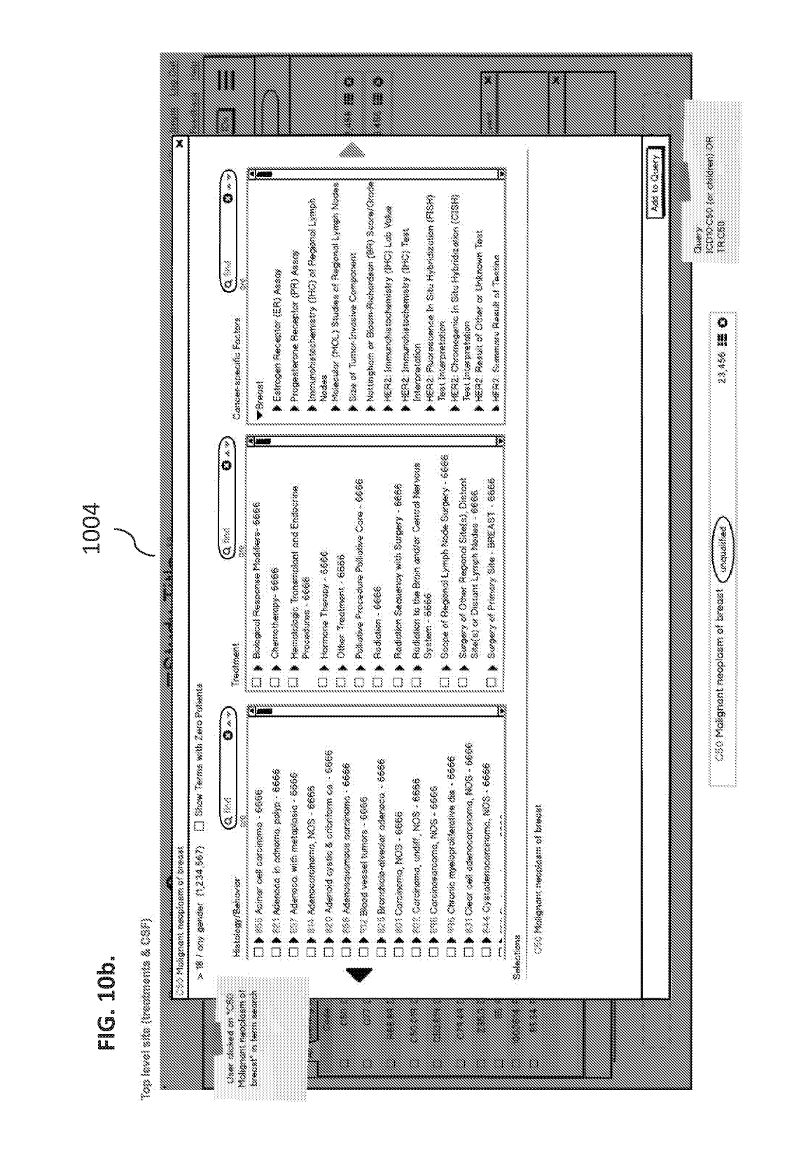

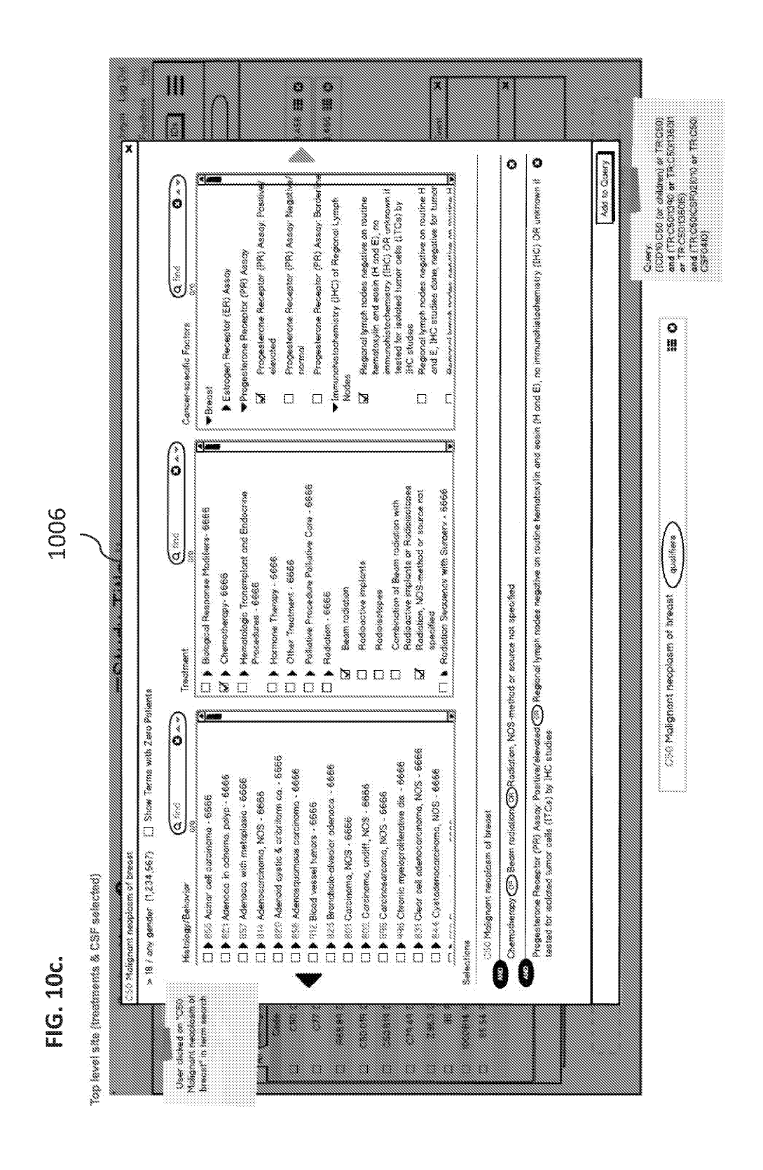

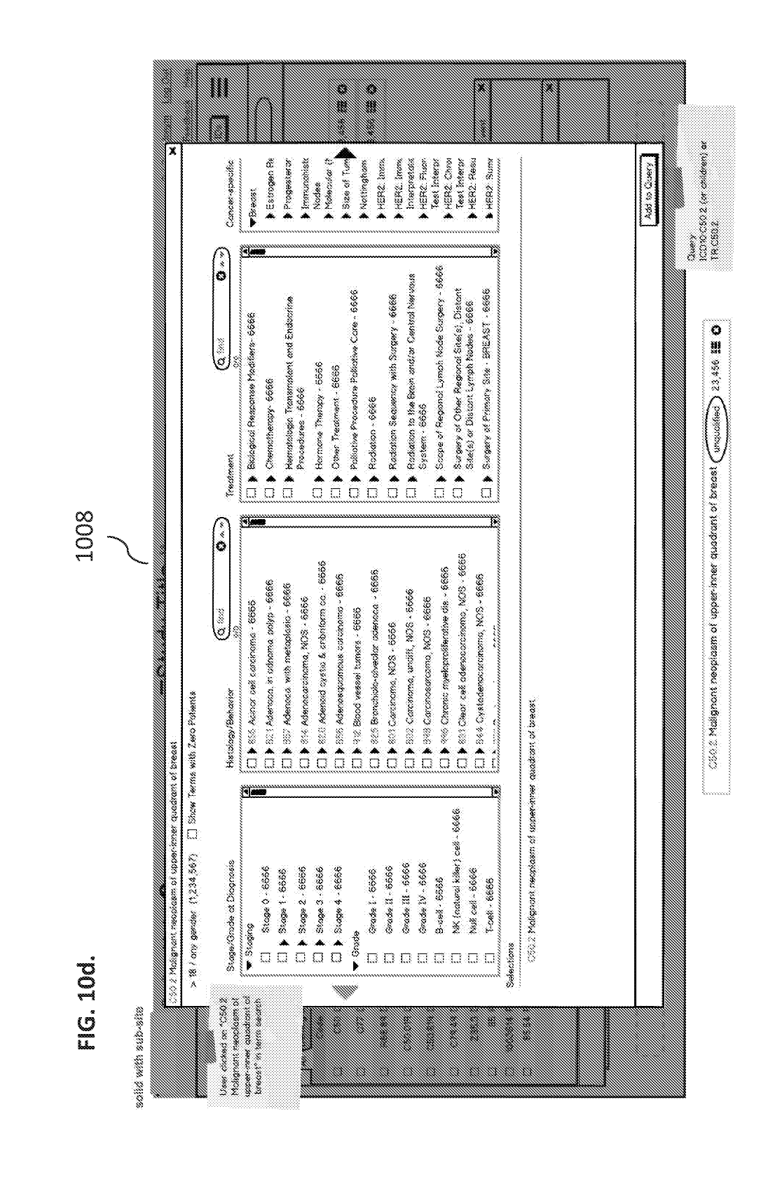

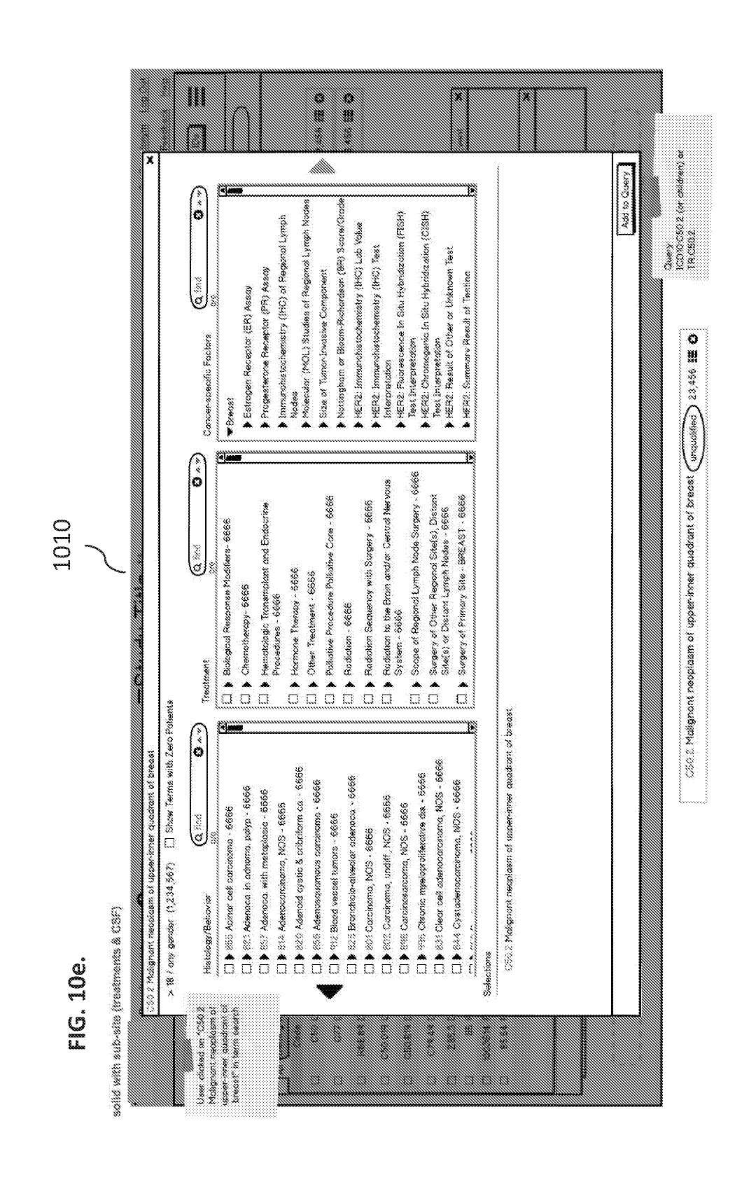

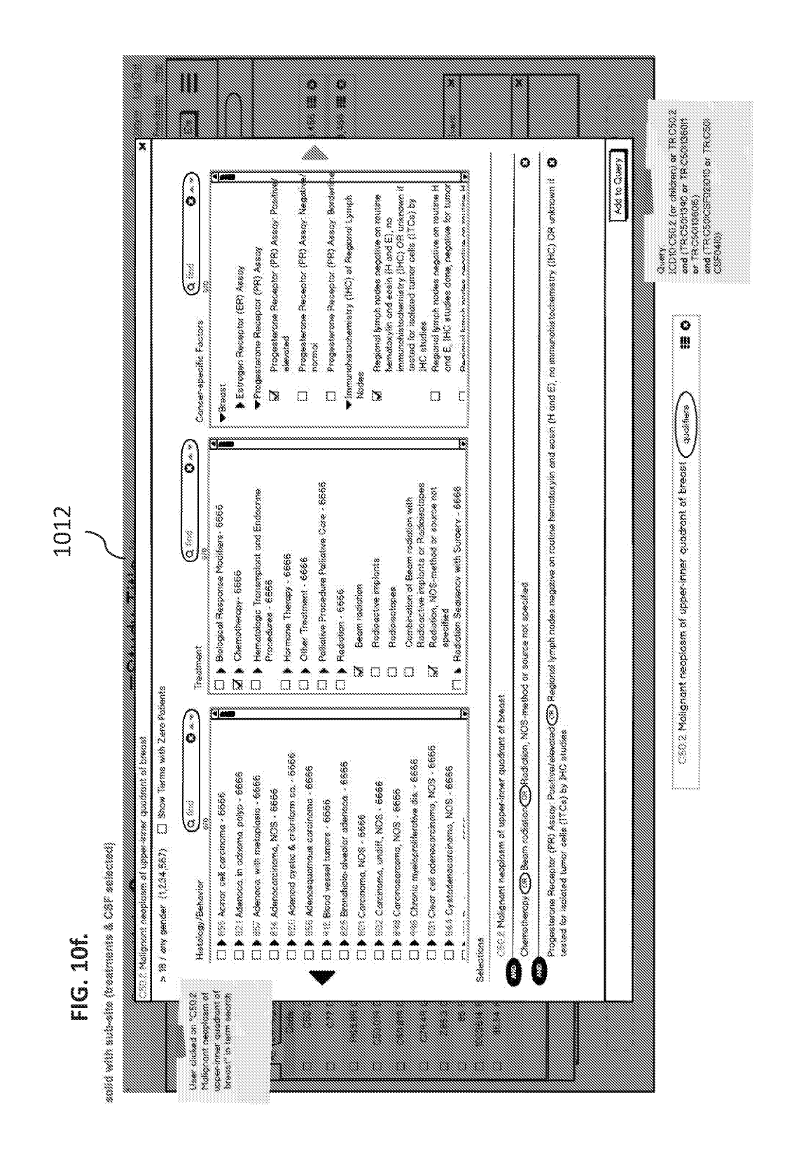

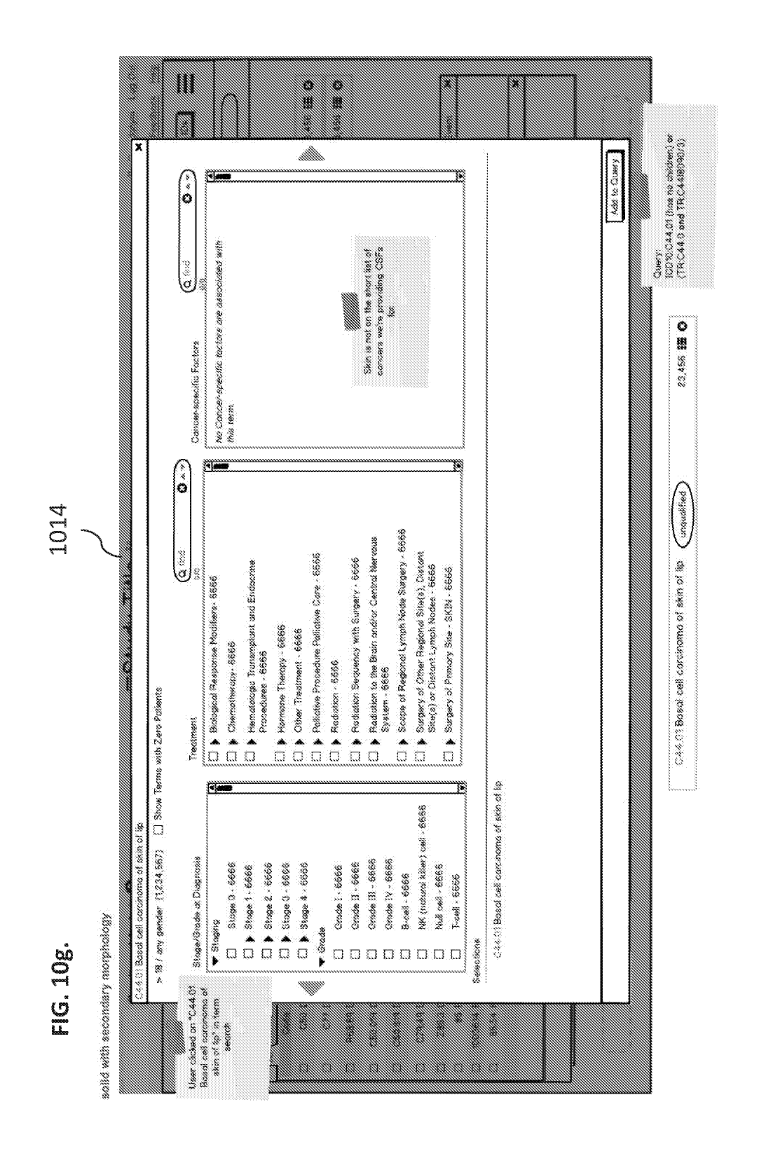

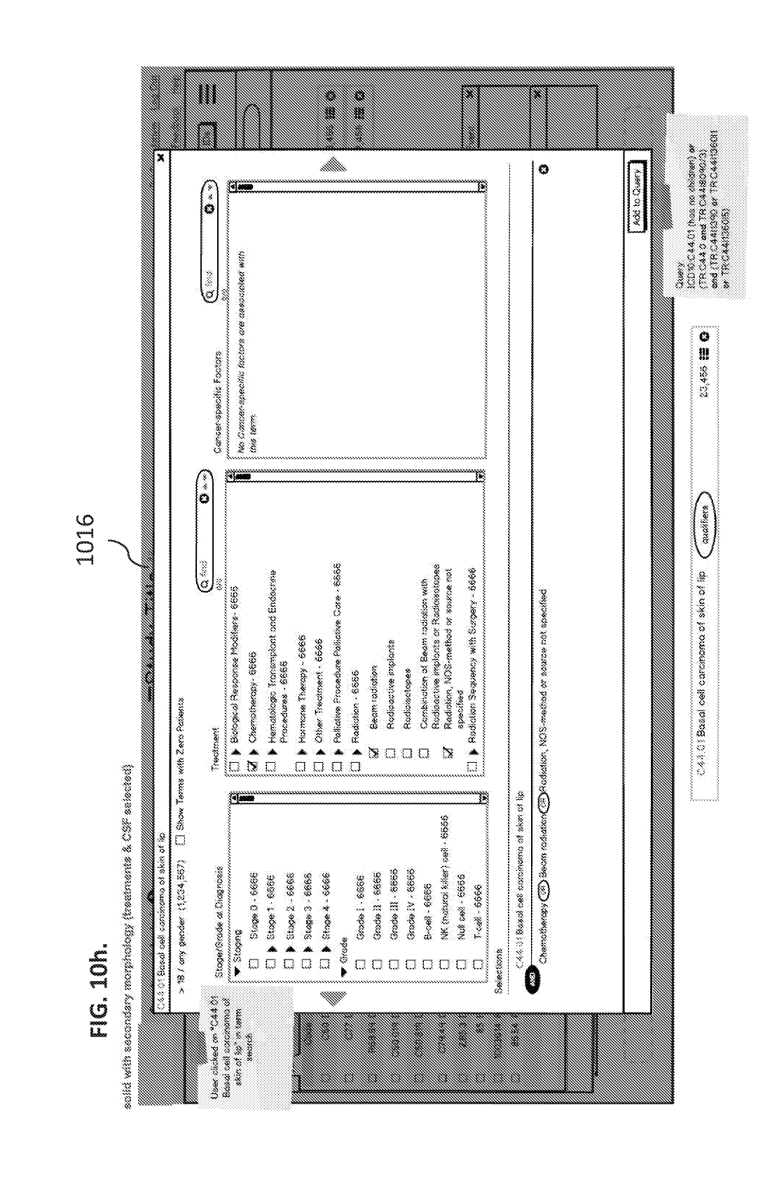

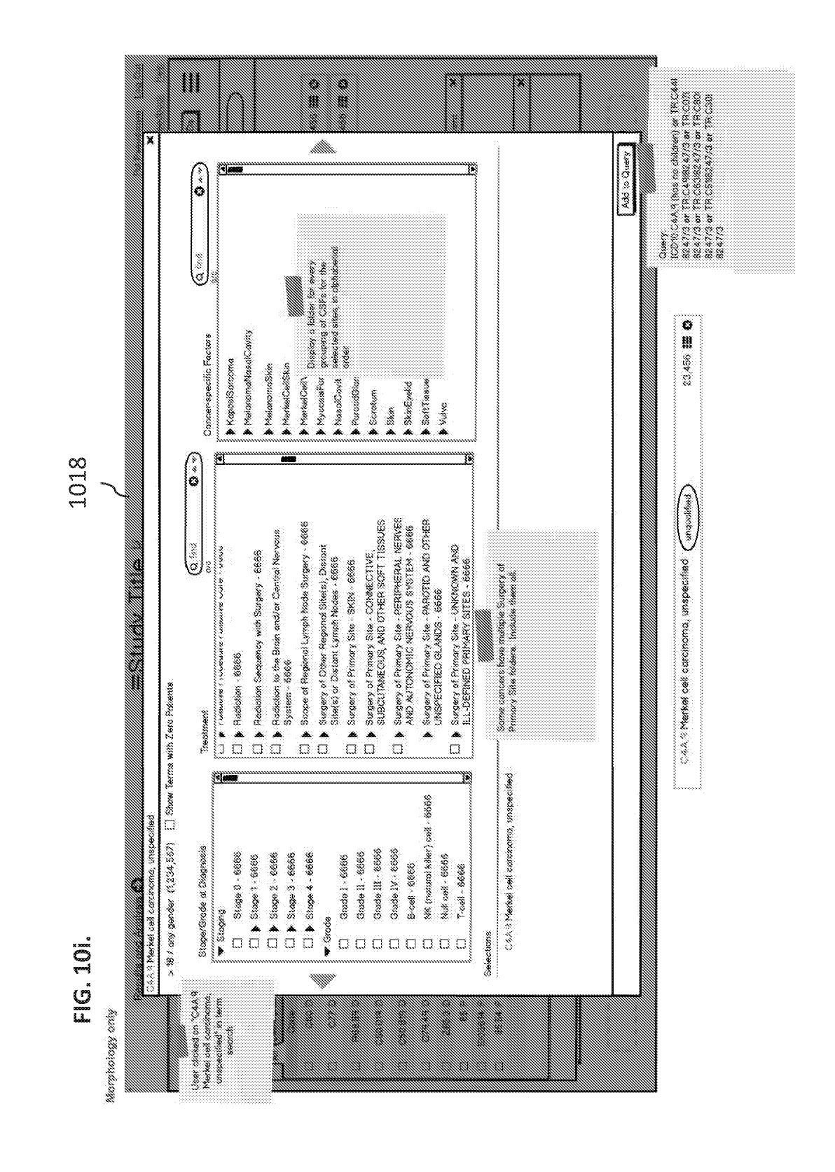

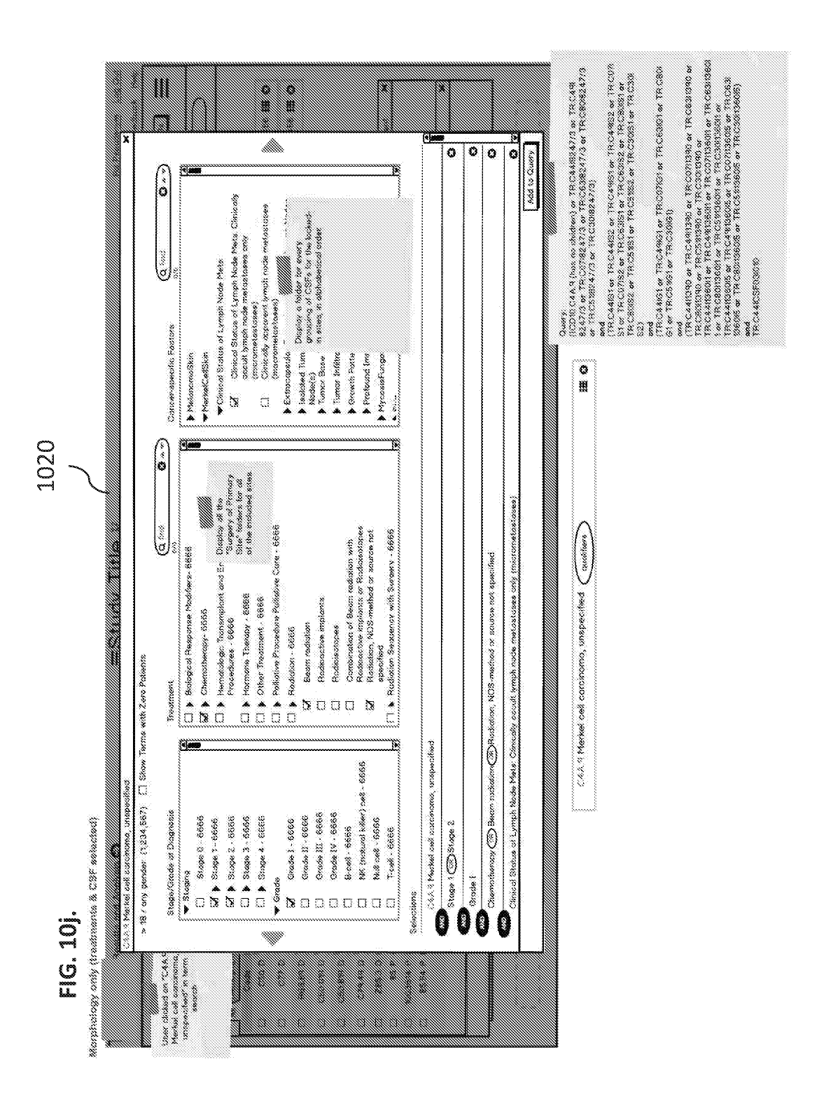

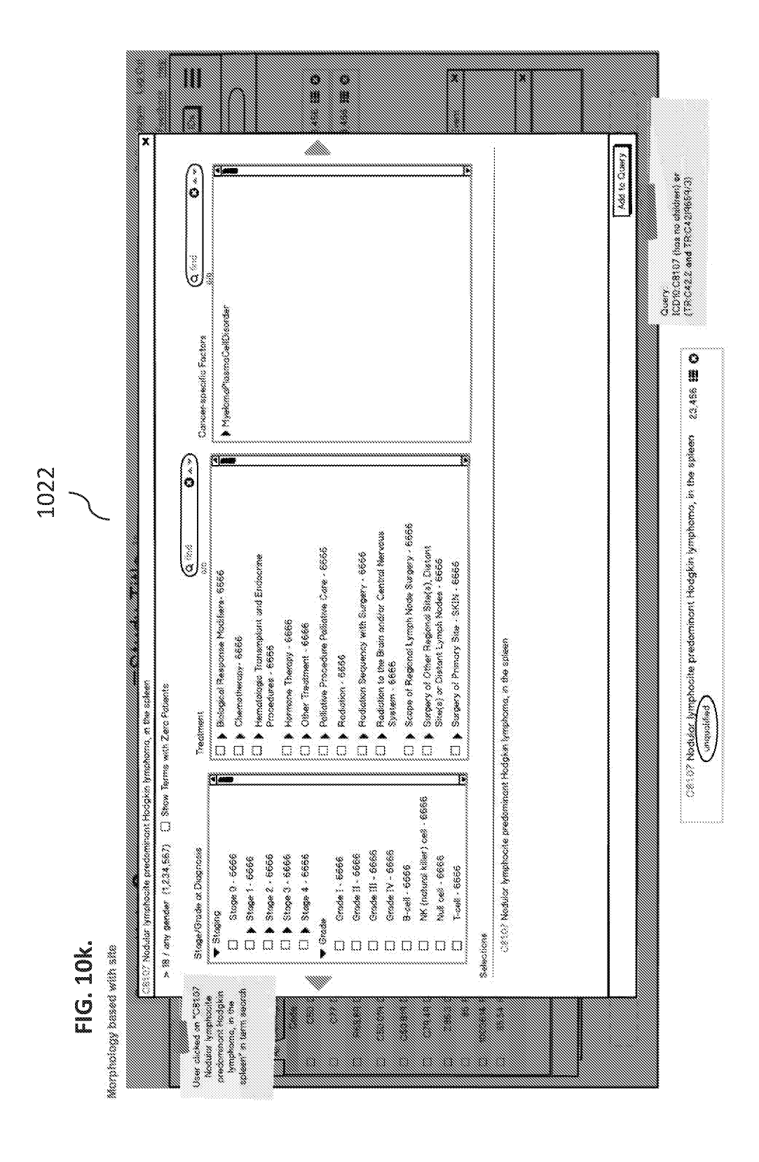

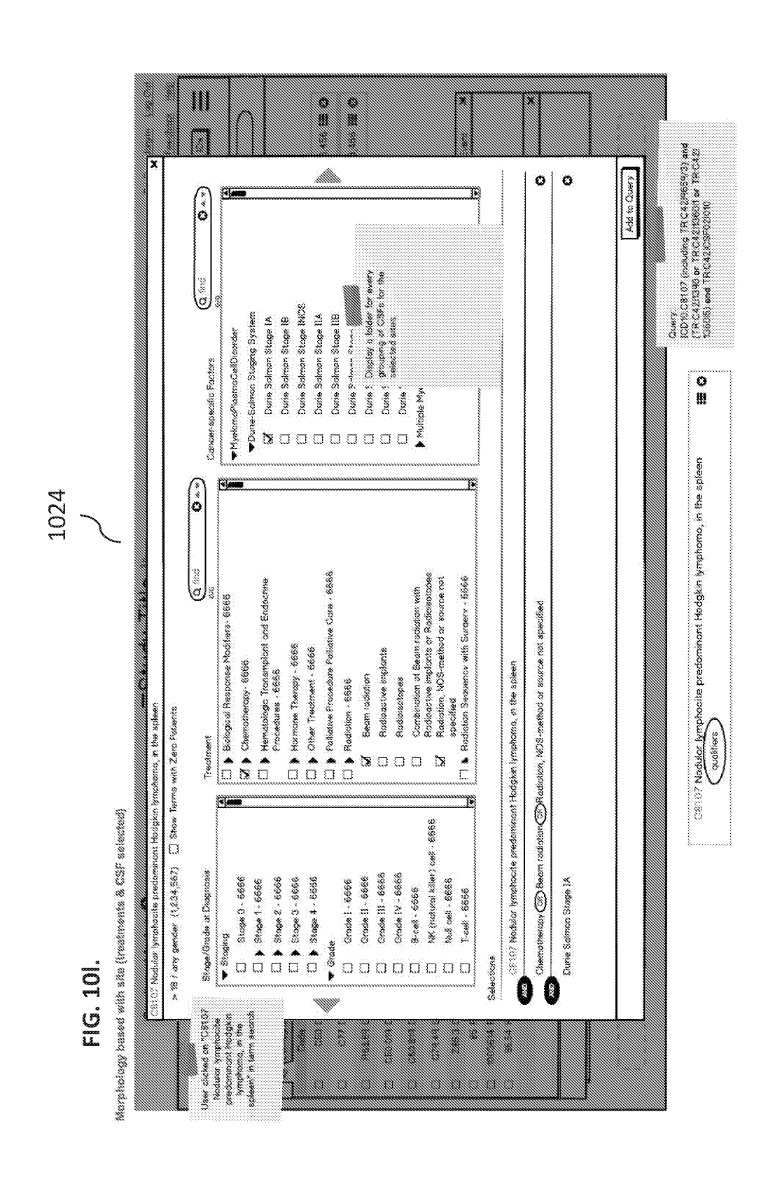

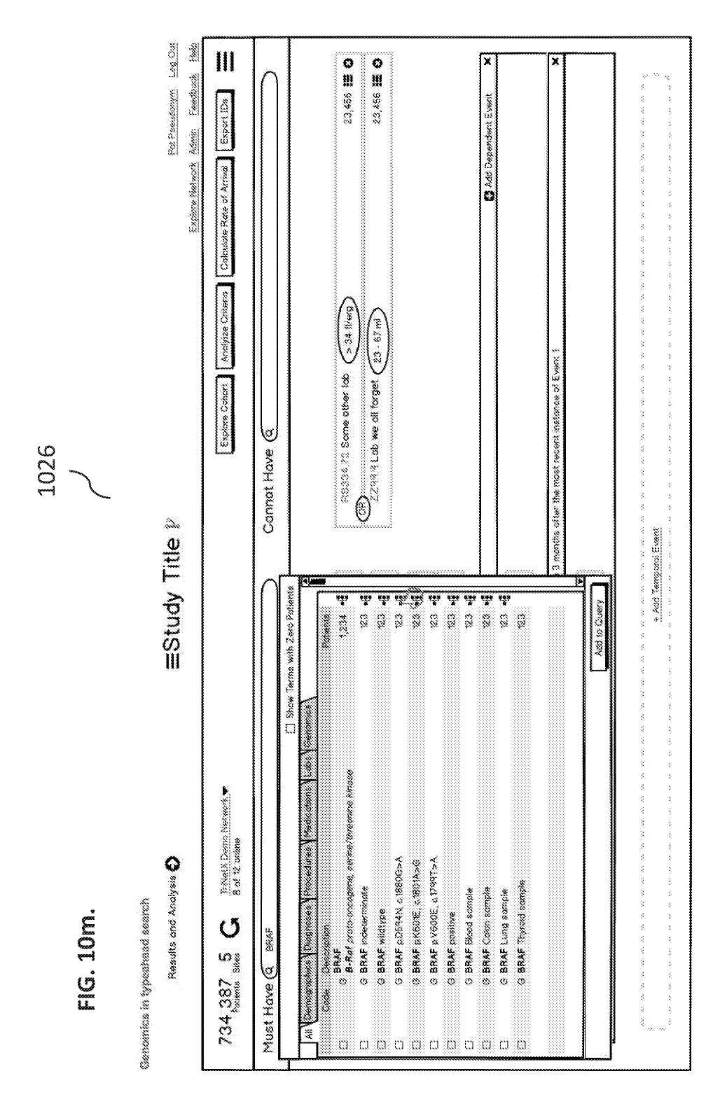

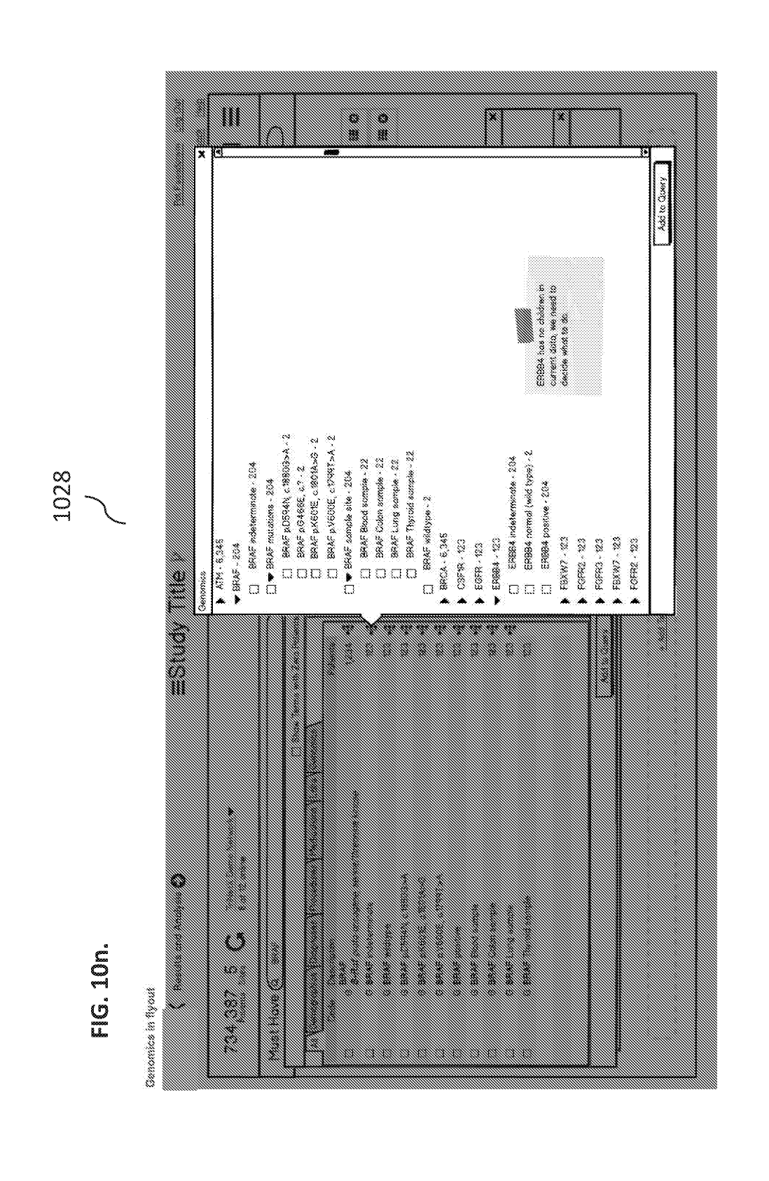

In some exemplary implementations, ICD-10-CM C00-D49 concepts can be mapped to an ICD-O site, an ICD-O morphology, and/or both (with indicator of whether site and/or morphology are the primary mapping). In some implementations, mappings can be enriched by: inheritance from ICD-10-CM children, known relationships from ICD-O morphologies to ICD-O sites, instance patient data, synonyms, and/or any other information. Choosing an ICD-10-CM diagnosis with an appropriate mapping can allow the user to further qualify the cancer with tumor registry-derived observations. Exemplary mappings are shown in FIGS. 10a-n.

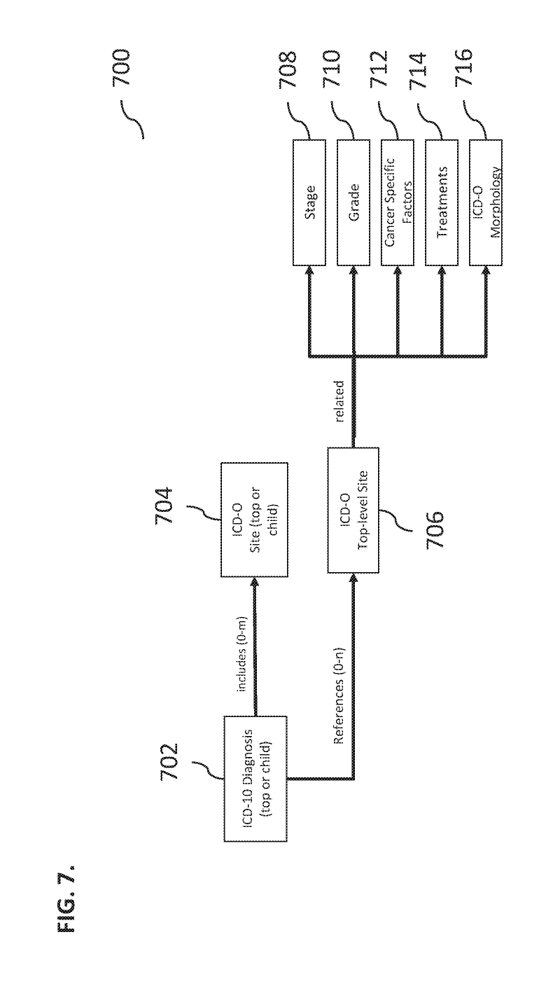

FIG. 7 illustrates an exemplary site-specific oncology data model 700, according to some implementations. The data model 700 can be used to generate a search query based on search terms that may have been entered by the user and/or supplied by the system (e.g., systems shown in FIGS. 1 and 3). The data model 700 can be stored, used and/or implemented by the system to generate a query for retrieval of data (e.g., data relating to a tumor diagnosis for a particular patient/patients, any cohort of patients, etc.).

In some implementations, the data model 700 can include a top level/node 702, dependent level nodes 704 and 706, where dependent level/node 706 can also have dependent levels/nodes 708-716. The top level node 702 can, for example, represent a top or a child level/node corresponding to an ICD-10 diagnosis. The node 704 can be also a top or a child level/node corresponding to an ICD-O site. It can be associated with the node 702 via an “include” relationship, e.g., the ICD-10 diagnosis can “include” one or more (e.g., 0−m, where m is an integer) ICD-O sites.

Further, the node 702 can be associated with the node 706 via a “reference” relationship. The node 706 can be a top-level site corresponding to, for example, an ICD-O top level site. This can mean that the ICD-10 diagnosis can have one or more references (e.g., 0-n, where n is an integer) to an ICD-O top-level site. As shown in Appendix A, the ICD-O is organized in a hierarchical structure, and thus, a top-level site can be representative of a particular level within that hierarchical structure to which the ICD-10 diagnosis 702 can have a “reference” to. Similarly, the ICD-O site 704 can be representative of a level within the hierarchical structure which the ICD-10 diagnosis 702 can “include”.

The ICD-O top level site node 706 can further be associated with nodes 708-716 via a “related” relationship. For example, the ICD-O top level site node 706 can be related to a stage node 708 (e.g., a stage of cancer), a grade node 710 (e.g., a grade of cancer), cancer specific factor(s) (“CSF”) node 712 (e.g., cancer specific factors associated with specific cancer diagnosis), treatment(s) node 714 (e.g., treatments that may have been performed and/or recommended for the patient(s) with a particular cancer diagnosis and/or cancer type, stage, grade, etc.), and an ICD-O morphology node 716.

Thus, when search terms for a query are received, the current subject matter system can generate a query that can correspond to the identifiers or codes associated with the ICD-10 diagnosis, which can “include” any identifiers or codes associated with the ICD-O site and/or “reference” an ICD-O top-level site identifiers, which, in turn, can include any “related” identifiers or codes associated with stage, grade, CSF, treatment(s), and/or ICD-O morphology. Further, upon selection of a particular ICD-10 diagnosis, the current subject matter can generate a query to automatically include other ICD-O types of information. This way the user does not have to automatically and/or manually add such ICD-O information. Thus, for the purposes of the query, the user may need to know ICD-10 coding schemes only. The “references” and “related” nodes can be used for generation of selected stage(s), grade(s), CSF(s), treatment(s), ICD-O morphology identifier(s) or code(s) 708-716 that can be included in the query. These can be pre-defined in the master terminology structure using the “included” site nodes, whereby the child nodes can be “walked” through to obtain the unique site identifiers/codes and/or truncate all site identifiers/codes to a 3-character level ICD-O site code. When generating a query, for each user-selected stage, grade, CSF, treatment, morphology identifiers/codes, a query term can be generated for each “reference” site 706. As stated above, the ICD-O top-level site(s) 706 can include “related” sub-level node(s): stage 708, grade 710, cancer-specific factors 712, treatments 714, and ICD-O morphology 716.

For example, assuming in the site-specific oncology data model 700, C50 is selected as the ICD-10 diagnosis node 702. Further, stage 2 (“S2”), stage 3 (“S3”), carcinoma NOS (“8010/2”), carcinoma in situ NOS (“8010/3”) are selected as child nodes (e.g., child nodes 708 and 712), the query to retrieve desired data can be generated in the following manner:
ICD-10:C50 or TR:C50 or ICD-10:C50.1 or TR:C50.1 or ICD-10:C50.2 or TR:C50.2 and TR:C50|S2 or TR:C50|S3

### and TR:C50|8010/2 or TR:C50|8010/3

In the above query, “ICD-10:C50”, “ICD-10:C50.1”, and “ICD-10:C50.2” can correspond to the ICD-10 diagnosis site, where “ICD-10:C50” can correspond to a top level and “ICD-10:C50.1” and “ICD-10:C50.2” can correspond to child levels (where “TR” is tumor registry). The “TR:C50”, “TR:C50.1” and “TR:C50.2” can correspond to the “included” ICD-O sites, where “TR:C50” can be the top “included” ICD-O site and “TR:C50.1” and “TR:C50.2” can correspond to the child “included” ICD-O sites. The reference ICD-O site is “TR:C50”, which can have “related” stage sites 708, i.e., “TR:C50|S2” or “TR:C50|S3”, and “related” CSF sites 712, i.e., “TR:C50|8010/2” or “TR:C50|8010/3”.

In some implementations, the current subject matter system can connect all child level nodes (e.g., C50.1, C50.2) and their “included” ICD-O (TR) site codes together using a Boolean OR operator, as shown in the above query. This can allow for an expanded search of data of not only the top level site (i.e., C:50), but also child nodes (i.e., C50.1, C50.2). Each selected stage and morphology term can be generated using the 3-character ICD-O (TR) site identifier/code. Each type can connected together using a Boolean AND operator, as shown above.

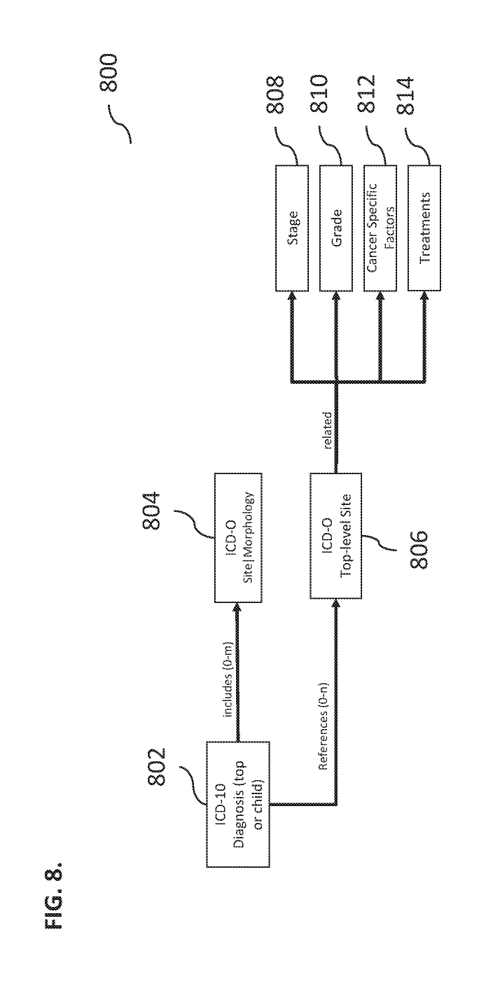

FIG. 8 illustrates an exemplary non-site-specific oncology data model 800, according to some implementations of the current subject matter. The data model 800, similar to data model 700 shown in FIG. 7, can be used to generate a search query based on search terms that may have been entered by the user and/or supplied by the system (e.g., systems shown in FIGS. 1 and 3). The data model 800 can represent a non-site specific oncology data model. The data model 800 can be stored, used and/or implemented by the system to generate a query for retrieval of data (e.g., data relating to a tumor diagnosis for a particular patient/patients).

In some implementations, the data model 800 can include a top level node 802, dependent level nodes 804 and 806, where dependent level node 806 can also have dependent level nodes 808-814. The top level node 802 can, for example, represent a top or a child level site corresponding to an ICD-10 diagnosis. The node 804 can be a site corresponding to an ICD-O|Morphology site. It can be associated with the node 802 via the “include” relationship, e.g., the ICD-10 diagnosis can “include” one or more (e.g., 0-m, where m is an integer) ICD-O|Morphology sites.

Further, the node 802 can be associated with the site/node 806 via a “reference” relationship. The node 806 can be a top-level site corresponding to, for example, an ICD-O top level site. This can mean that the ICD-10 diagnosis can have one or more references (e.g., 0−n, where n is an integer) to an ICD-O top-level site. As stated above, the top-level site can be representative of a particular level within that hierarchical structure (as shown in Appendix A) to which the ICD-10 diagnosis 802 can have a “reference” to.

Similar to the model 700 shown in FIG. 7, the ICD-O top level site 806 can further be associated with nodes 808-814 via a “related” relationship. The ICD-O top level site node 806 can be related to a stage node 808, a grade node 810, CSF node 812, and treatment(s) node 814. The morphology information (shown in the model 700 as being “related” to the ICD-O top level site) is incorporated into the ICD-O node 804, as the model 800 is non-site specific.

Similar to model 700, when search terms for a query are received, the current subject matter system can generate a query that can include identifiers/codes corresponding to the ICD-10 diagnosis, which can “include” any identifiers/codes corresponding to the ICD-O|Morphology site and/or “reference” the ICD-O top-level site identifiers, which, in turn, can include any “related” identifiers/codes corresponding to the stage, grade, CSF, and treatment(s). When a particular ICD-10 diagnosis is selected, the current subject matter can generate a query to include other ICD-O|Morphology information. This way the user does not have to automatically and/or manually add it. Thus, similar to the model 700, the user may need to know ICD-10 coding schemes only. The “references” and “related” nodes can be used for generation of selected stage(s), grade(s), CSF(s), and treatment(s) identifier(s)/code(s) 808-814 that can be included in the query. These can be pre-defined in the master terminology structure using the “included” site nodes, whereby the child nodes can be “walked” through to obtain the unique site identifiers/codes and/or truncate all site identifiers/codes to a 3-character level ICD-O site code. When generating a query, for each user-selected stage, grade, CSF, treatment identifiers/codes, a query term can be generated for each “reference” site 806. As stated above, the ICD-O top-level site(s) 806 can include “related” sub-level node(s): stage 808, grade 810, cancer-specific factors 812, and treatments 814.

For example, a query for a Hodgkin's disease with a user-selected stage 2 can be represented as follows:
ICD-10:C81.0 or ICD-10:C81.00 or ICD-10:C81.01 or ICD-10:C81.02 or ICD-10:C81.03 or ICD-10:C81.04 or ICD-10:C81.05 or ICD-10:C81.06 or ICD-10:C81.07 or ICD-10:C81.0b or ICD-10:C81.09 or TR:C42|9659/3 or TR:C77|9659/3

### and TR:C77|S2 or TR:C42|S2

In the above query, “ICD-10:C81.0” has been identified as an ICD-10 diagnosis or a top level site, which in this case C81 corresponds to Hodgkin lymphoma ICD-10 diagnosis. This identifier/code can correspond to a search term that may have been submitted to the current subject matter system (e.g., systems 100, 300, as shown in FIGS. 1, 3). The current subject matter can execute a process whereby the entered terms are converted to specific identifiers/codes. Alternatively, a particular ICD-10 diagnosis/code can be presented to the current subject matter system. Based on the top level diagnosis, the current subject matter system can identify all relevant child nodes (e.g., by searching through the ICD-10 hierarchical data structure). In the above query, the child nodes can include “ICD-10:C81.00”, “ICD-10:C81.01”, “ICD-10:C81.02”, “ICD-10:C81.03”, “ICD-10:C81.04”, “ICD-10:C81.05”, “ICD-10:C81.06”, “ICD-10:C81.07”, “ICD-10:C81.0b”, and “ICD-10:C81.09”. As shown above, these top node and the child nodes can be connected by a Boolean OR operator.

The current subject matter's system can also convert the entered/provided search terms to “include” an ICD-O site|morphology identifiers/codes of “TR:C42|9659/3” and “TR:C77|9659/3”. These codes can again be connected using a Boolean OR operator.

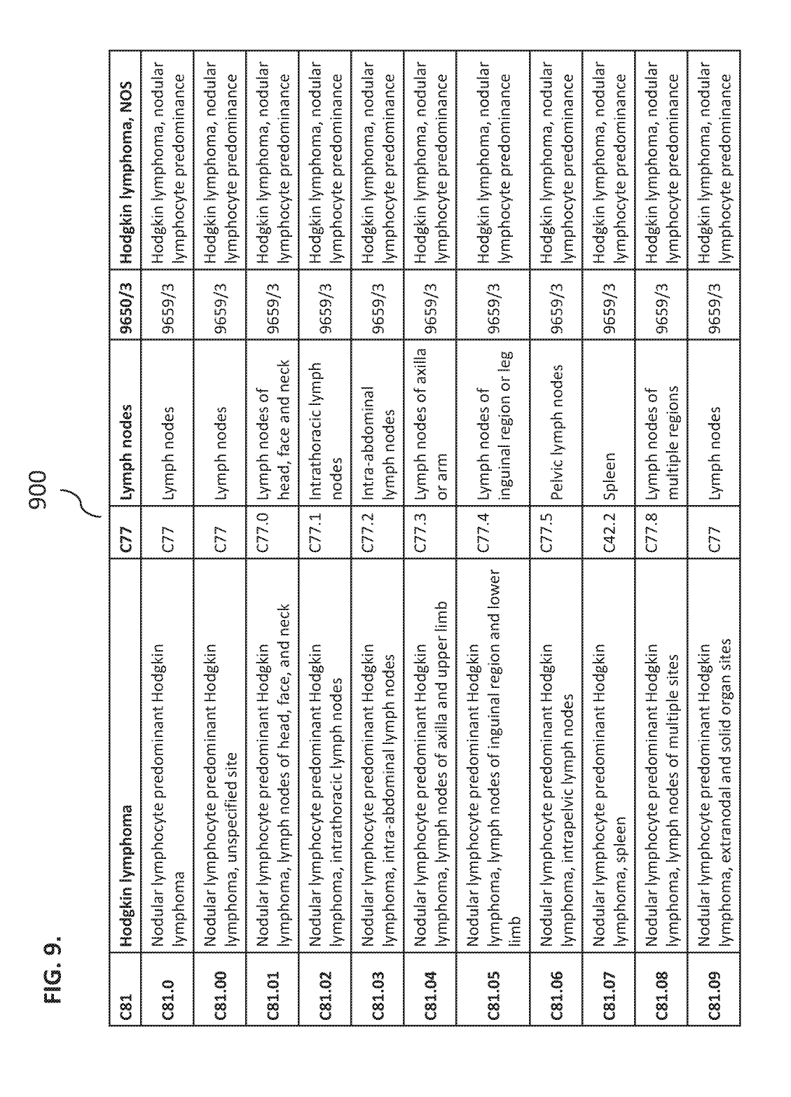

In this query, no specific ICD-O site has been identified and instead, only a particular stage (i.e., “stage 2” or “S2”) has been selected as being of interest. Thus, the current subject matter's system determines identifiers/codes that are indicative of the particular stage as relating to the ICD-O site|morphology and determined based on the ICD-10 diagnosis codes. As shown in the above query, the identifiers/codes indicative of the stage are “TR:C77|S2” and “TR:C42|S2”. The identifiers/codes can be connected to each other via a Boolean OR operator and to the remainder of query using a Boolean AND operator. FIG. 9 illustrates an exemplary table 900 showing identification of identifiers/codes corresponding to the query above.

Additional exemplary queries containing mappings are illustrated as Scenarios 1-4 in Appendix B.

In some implementations, the current subject matter can relate to a tumor terminology structure or tumor registry (“TR”) hierarchy in a format of i2b2 ontology. The TR hierarchy can be a multi-level hierarchy and can be arranged as follows:
Level 0—“Tumor Registry”
   Level 1—“Sites” (or any other parameters)
      Level 2—custom overlay by clinical oncology
         Level 3—ICD-O topology, top-level (C## format)
         Level 4:
         ICD-O topology sub-sites
         Stage/TNM
         Grade
         Histology/Behavior
         Cancer-Specific Factors (CSF)
         Treatment

The current subject matter's system, upon receiving a search request or a query that can include various search terms, can execute a process whereby search terms can be analyzed and specific identifiers/codes can be determined and/or identified in accordance with the above procedures. The system can perform a search of a hierarchy of the identifiers/codes in various registries and extract appropriate identifiers/codes for the purposes of creating a mapping between determined/identified identifiers/codes. Once the identifiers/codes are determined/identified, a mapping can be created (e.g., similar to the models 700 and 800, as shown in FIGS. 7 and 8, respectively). The created mapping can be used to generate a query to one or more databases containing data (e.g., data relating to various cancer and/or any other medical conditions cases). The current subject matter's system can submit the query to the databases for searching and identifying data that is responsive to the entered search terms. The query can be submitted over a network, e.g., the Internet, intranet, extranet, WAN, LAN, MAN, VLAN, etc. Once the data responsive to the query has been identified, it can be transmitted to for a display on one or more user interfaces. The data can be formatted and/or graphically arranged on the user interface(s).

FIGS. 10a-n illustrate various interfaces 1002-1028, according to some implementations of the current subject matter. FIG. 10a illustrates an interface 1002 showing a top level site corresponding to “C50 Malignant neoplasm of breast”. The following query can be added to display all available results for this top level site:
ICD-10:C50 (or children) or TR:C50

The interface 1002 can also display all available stage, grade, histology/behavior, treatment, CSF, etc. parameters that can be selected or selectable for the purposes of limiting the query and/or data responsive to the query. For example, some parameters, e.g., staging and grade, can be shown in an expanded form in the interface 1002, while others, e.g., histology/behavior, treatment, CSF, can be shown in a collapsed form in the interface 1002. Each particular parameter can be graphically expanded to show sub-categories, which can be selected. Selection can be performed automatically and/or manually, e.g., using a mouse, a keyboard, a stylus pen, etc. by clicking on an action box next to a particular parameter.

FIG. 10b illustrates an interface 1004 showing the top level site as shown in the interface 1002 together with the histology/behavior, treatment, and CSF. The same query shown in the interface 1002 can be added to display all available results for this top level site. The user can be allowed to scroll through all parameters that may be associated with this top level site (i.e., C50). The scrolling can be performed automatically and/or manually, e.g., using a mouse, a keyboard, a stylus pen, etc.

FIG. 10c illustrates an interface 1006 showing a top level site corresponding to “C50 Malignant neoplasm of breast” with certain treatments and CSF selected. The following query can be used for such selection:
(ICD-10:C50 (or children) or TR:C50) and
(TR:C50|1390 or TR:C50|1360|/1 or TC:C50|1360|5) and
(TR:C50|CSF02|010 or TR:C50|CSF04|0)

This query can correspond to the following parameters “C50 Malignant neoplasm of breast” AND (a Boolean operator) treatment(s) parameter (i.e., “Chemotherapy” (i.e., a treatment corresponding to “TR:C50|1390”) OR (a Boolean operator) “Beam Radiation” (i.e., a treatment corresponding to “TR:C50|136011” OR “Radiation, NOS-method or source not specified” (i.e., a treatment corresponding to “TC:C50|136015”)) AND CSF parameter(s) (i.e., “Progesterone Receptor (PR) Assay: Positive/Elevated” (i.e., a CSF corresponding to “TR:C50|CSF02|010”) OR “Regional lymph nodes negative on routine hematoxylin and eosin (H and E), no immunohistochemistry (IHC) OR unknown if tested for isolated tumor cells (ITCs) by IHC studies” (i.e., a CSF corresponding to “TR:C50|CSF04|0”)). As shown in FIG. 10c, appropriate graphical checkboxes contained in the interface 1006 have been checked corresponding to the above selections.

FIG. 10d illustrates an interface 1008 showing a sub-site corresponding to “C50.2 Malignant neoplasm of upper-inner quadrant of breast”. The following query can be added to display all available results for this top level site:
ICD-10:C50.2 (or children) or TR:C50.2

Similar to the interface 1002, the interface 1008 can also display all available stage, grade, histology/behavior, treatment, CSF, etc. parameters that can be selected or selectable for the purposes of limiting the query and/or data responsive to the query. FIG. 10e illustrates an interface 1010 showing the sub-site as shown in the interface 1008 together with the histology/behavior, treatment, and CSF. The same query shown in the interface 1008 can be added to display all available results for this sub-site. The user can be allowed to scroll through all parameters that may be associated with this sub-site (i.e., C50.2). The scrolling can be performed automatically and/or manually, e.g., using a mouse, a keyboard, a stylus pen, etc.

FIG. 10f illustrates an interface 1012 the sub-site corresponding to “C50.2 Malignant neoplasm of upper-inner quadrant of breast” (as shown in FIGS. 10d-e) with certain treatments and CSF selected. The following query can be used for such selection:
(ICD-0: C50.2 (or children) or TR: C50.2) and
(TR:C50.2|1390 or TR:C50.2|1360|1 or TC:C50.2|1360|5) and
(TR:C50.2|CSF02|010 or TR:C50.2 CSF04|0)

This query is similar to the query shown in FIG. 10c but is being performed on the sub-site (i.e., C50.2). Again similar to the query in FIG. 10c, the query shown in the interface 1012 can correspond to the following parameters “C50.2 Malignant neoplasm of upper-inner quadrant of breast” AND (a Boolean operator) treatment(s) parameter (i.e., “Chemotherapy” (i.e., a treatment corresponding to “TR:C50.2|1390”) OR (a Boolean operator) “Beam Radiation” (i.e., a treatment corresponding to “TR:C50.2|1360|1” OR “Radiation, NOS-method or source not specified” (i.e., a treatment corresponding to “TC:C50.2|1360|5”)) AND CSF parameter(s) (i.e., “Progesterone Receptor (PR) Assay: Positive/Elevated” (i.e., a CSF corresponding to “TR:C50.2|CSF02|010”) OR “Regional lymph nodes negative on routine hematoxylin and eosin (H and E), no immunohistochemistry (IHC) OR unknown if tested for isolated tumor cells (ITCs) by IHC studies” (i.e., a CSF corresponding to “TR:C50.2|CSF04|0”)). As shown in FIG. 10f, appropriate graphical checkboxes contained in the interface 1012 have been checked corresponding to the above selections.

FIG. 10g illustrates an interface 1014 showing a site with secondary morphology corresponding to “C44.01 Basal cell carcinoma of skin of lip” being selected (e.g., by a user). The following query can be added to display all available results for this top level site:
ICD-10:C44.01 (has no children) or (TR:C44.01 and TR:C44|8090/3)

The interface 1014 can also display windows for all available stage/grade at diagnosis, treatment, and CSF parameter that can be selected or selectable for the purposes of limiting the query and/or data responsive to the query. Some parameters might not be available for selection (e.g., CSF). Further, some parameters, e.g., staging/grade at diagnosis, can be shown in an expanded form in the interface 1014, while others, e.g., treatment, can be shown in a collapsed form in the interface 1014. Each particular parameter can be graphically expanded to show sub-categories, which can be selected. Selection can be performed automatically and/or manually, e.g., using a mouse, a keyboard, a stylus pen, etc. by clicking on an action box next to a particular parameter.

FIG. 10h illustrates an interface 1016 showing a site with secondary morphology corresponding to “C44.01 Basal cell carcinoma of skin of lip”, as shown in FIG. 10g, with certain treatments and CSF being selected. The following query can be used for such selection:
ICD-10:C44.01 (has no children) or (TR:C44.01 and TR:C44|8090/3) and
(TR:C44.0 or TR:C44|1360|1 or TR:C44|1360|5)

This query can correspond to the following parameters “C44.01 Basal cell carcinoma of skin of lip” (i.e., ICD-10:C44.01 (has no children) or (TR:C44.01 and TR:C44|8090/3)) AND (a Boolean operator) treatment(s) parameter (i.e., “Chemotherapy” (i.e., a treatment corresponding to “TR:C44.0”) OR “Beam Radiation” (i.e., a treatment corresponding to “TR:C44|136011” OR “Radiation, NOS-method or source not specified” (i.e., a treatment corresponding to “TC:C44|136015”)). As shown in FIG. 10h, appropriate graphical checkboxes contained in the interface 1016 have been checked corresponding to the above selections.

FIG. 10i illustrates an interface 1018 showing morphology only corresponding to “C4A.9 Merkel cell carcinoma, unspecified” being selected. The following query can be added to display all available results for this top level site:
ICD-10:C4A.9 (has no children) or TR:C44|8247/3 or TR:C49|8247/3 or TR:C07|8247/3 or TR:C63|8247/3 or TR:C80|8247/3 or TR:C51|8247/3 or TR:C30|8247/3

The interface 1018 can also display windows for all available stage/grade at diagnosis, treatment, and CSF parameters that can be expanded/selected/selectable for the purposes of limiting the query and/or data responsive to the query. Some parameters might not be available for selection (e.g., CSF), as, for example, not being included in a particular ICD-10 parameter. Further, some parameters, e.g., staging/grade at diagnosis, can be shown in an expanded form in the interface 1018, while others, e.g., treatment, can be shown in a collapsed form in the interface 1018. Each particular parameter can be graphically expanded to show sub-categories, which can be selected. Selection can be performed automatically and/or manually, e.g., using a mouse, a keyboard, a stylus pen, etc. by clicking on an action box next to a particular parameter.

FIG. 10j illustrates an interface 1020 that is based on the interface 1018 shown in FIG. 10i, where certain treatments and CSF are selected for the query. The following query can be used for such selection:
(ICD-10:C4A.9 (has no children) or TR:C44|8247/3 or TR:C49|8247/3 or TR:C07|8247/3 or TR:C63|82473 or TR:C80|8247/3 or TR:C51|8247/3 or TR:C30|8247/3) and
(TR:C44|S1 or TR:C441|S2 or TR:C49|S1 or TR:C49|S2 or TR:C07|S1 or TR:C07|S2 or TR:C63|S1 or TR:C63|S2 or TR:C80|S1 or TR:C80|S2 or TR:C51|S1 or TR:C51|S2 or TR:C30|S1 or TR:C30|S2) and
(TR:C44|G1 or TR:C49|G1 or TR:C07|G1 or TR:C63|G1 or TR:C80|G1 or TR:C51|G1 or TR:C30|G1) and
(TR:C44|1390 or TR:C49|1390 or TR:C07|1390 or TR:C63|1390 or TR:C80|1390 or TR:C51|1390|1 or TR:C30|1390 or TR:C44|1360|1 or TR:C49|1360|1 or TR:C07|1360|1 or TR:C63|1360|1 or TR:C80|1360|1 or TR:C51|1360|1 or TR:C30|1360|1 or TR:C44|1360|5 or TR:C49|1360|5 or TR:C07|1360|5 or TR:C63|1360|5 or TR:C80|1360|5 or TR:C51|1360|5 or TR:C30|1360|5) and
TR:C44|CSF03|010

This query can correspond to the following parameters: “C4A.9 Merkel cell carcinoma, unspecified” (i.e., “ICD-10:C4A.9 (has no children) OR TR:C44|8247/3 OR TR:C49|8247/3 OR TR:C07|8247/3 OR TR:C63|8247/3 OR TR:C80|8247/3 OR TR:C51|8247/3 OR TR:C30|8247/3”) AND stage parameter (i.e., “stage 1” or “stage 2” (i.e., stages corresponding to “TR:C44|S1 OR TR:C44|S2 OR TR:C49|S1 OR TR:C49|S2 OR TR:C07|S1 OR TR:C07|S2 OR TR:C63|S1 OR TR:C63|S2 OR TR:C80|S1 OR TR:C80|S2 OR TR:C51|S1 OR TR:C51|S2 OR TR:C30|S1 OR TR:C30|S2”)) AND grade parameter (i.e., “Grade 1” (i.e., a grade parameter corresponding to “TR:C44|G1 OR TR:C49|G1 OR TR:C07|G1 OR TR:C63|G1 OR TR:C80|G1 OR TR:C51|G1 OR TR:C30|G1”)) AND treatment(s) parameters (i.e., “Chemotherapy” (i.e., a treatment corresponding to “TR:C44|1390 OR TR:C49|1390 OR TR:C07|1390 OR TR:C63|1390 OR TR:C80|1390 OR TR:C51|1390 OR TR:C30|1390”) OR “Beam Radiation” (i.e., a treatment corresponding to “TR:C44|360|1 OR TR:C49|360|1 OR TR:C07|1360|1 OR TR:C63|1360|1 OR TR:C80|360|1 OR TR:C51|1360|1 OR TR:C30|1360|1”) OR “Radiation, NOS-method or source not specified” (i.e., a treatment corresponding to “TR:C44|1360|5 OR TR:C49|1360|5 OR TR:C07|1360|5 OR TR:C63|1360|5 OR TR:C80|1360|5 OR TR:C51|1360|5 OR TR:C30|1360|5”)) AND a CSF parameter (i.e., “Clinical Status of Lymph Node Mets: Clinically occult lymph node metastases only (micrometastases)” (i.e., “TR:C44|CSF03|010”)). As shown in FIG. 10j, appropriate graphical checkboxes contained in the interface 1020 have been checked corresponding to the above selections.

FIG. 10k illustrates an interface 1022 showing morphology based with site corresponding to “C81.07 Nodular lymphocyte predominant Hodgkin lymphoma, in the spleen” being selected. The following query can be added to display all available results for this top level site:
ICD-10:C81.07 (has no children) or (TR:C42.2 and TR:C42|9659/3)

Similar to other interfaces discussed above, the interface 1022 can also display windows for all available stage/grade at diagnosis, treatment, and CSF parameters that can be expanded/selected/selectable for the purposes of limiting the query and/or data responsive to the query. Some parameters, e.g., staging/grade at diagnosis, can be shown in an expanded form in the interface 1022, while others, e.g., treatment, CSF, can be shown in a collapsed form in the interface 1022. Each particular parameter can be graphically expanded to show sub-categories, which can be selected. Selection can be performed automatically and/or manually, e.g., using a mouse, a keyboard, a stylus pen, etc. by clicking on an action box next to a particular parameter.

FIG. 10l illustrates an interface 1024 that is based on the interface 1022 shown in FIG. 10k, where certain treatments and CSF are selected for the query. The following query can be used for such selection
ICD-10:C81.07 (including TR:C42|9659/3) and (TR:C42|1390 or TR:C42|1360|1 or TR:C42|1360|5 and TR:C42|CSF02|010)

This query can correspond to the following parameters “C81.07 Nodular lymphocyte predominant Hodgkin lymphoma, in the spleen” (i.e., ICD-10:C81.07 (including TR:C42|9659/3) AND treatment(s) parameter (i.e., “Chemotherapy” (i.e., a treatment corresponding to “TR:C42|1390”) OR “Beam Radiation” (i.e., a treatment corresponding to “TR:C42|1360|1” OR “Radiation, NOS-method or source not specified” (i.e., a treatment corresponding to “TC:C42|1360|5”)) AND CSF parameter(s) (i.e., “Durie Salmon Stage IA” (i.e., a CSF corresponding to “TR:C42|CSF02|010”)). As shown in FIG. 10l, appropriate graphical checkboxes contained in the interface 1024 have been checked corresponding to the above selections.

FIGS. 10m-n illustrate interfaces 1026 and 1028 that can allow the user to further specify information that must be included in the data that is being searched using the queries discussed above (e.g., blood sample, colon sample, etc.).

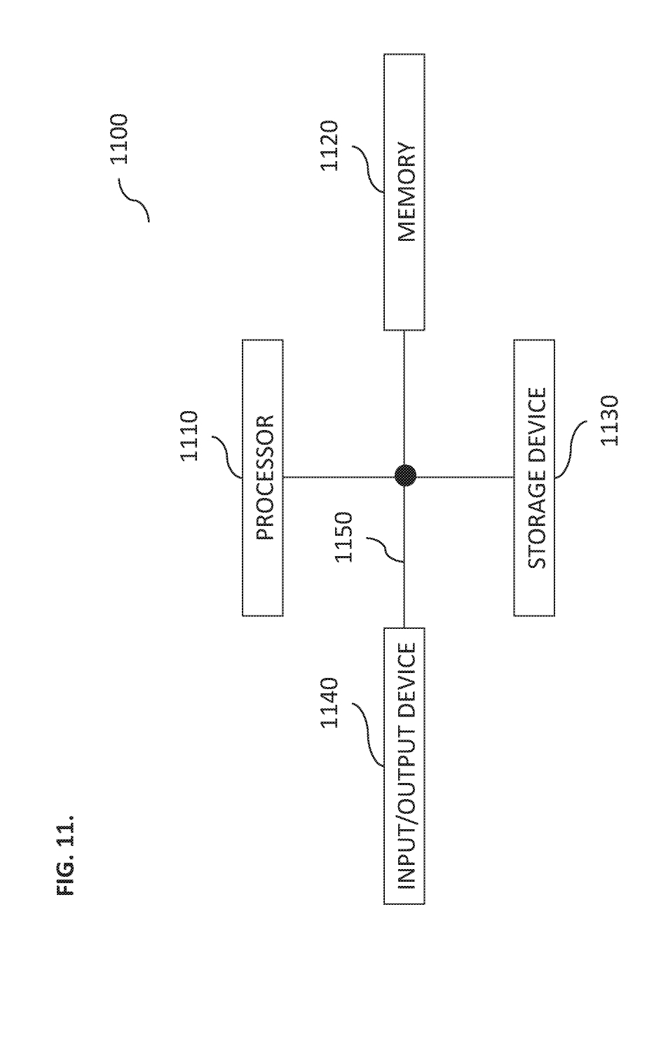

In some implementations, the current subject matter can be configured to be implemented in a system 1100, as shown in FIG. 11. The system 1100 can include a processor 1110, a memory 1120, a storage device 1130, and an input/output device 1140. Each of the components 1110, 1120, 1130 and 1140 can be interconnected using a system bus 1150. The processor 1110 can be configured to process instructions for execution within the system 1100. In some implementations, the processor 1110 can be a single-threaded processor. In alternate implementations, the processor 1110 can be a multi-threaded processor. The processor 1110 can be further configured to process instructions stored in the memory 1120 or on the storage device 1130, including receiving or sending information through the input/output device 1140. The memory 1120 can store information within the system 1100. In some implementations, the memory 1120 can be a computer-readable medium. In alternate implementations, the memory 1120 can be a volatile memory unit. In yet some implementations, the memory 1120 can be a non-volatile memory unit. The storage device 1130 can be capable of providing mass storage for the system 1100. In some implementations, the storage device 1130 can be a computer-readable medium. In alternate implementations, the storage device 1130 can be a floppy disk device, a hard disk device, an optical disk device, a tape device, non-volatile solid state memory, or any other type of storage device. The input/output device 1140 can be configured to provide input/output operations for the system 1100. In some implementations, the input/output device 1140 can include a keyboard and/or pointing device. In alternate implementations, the input/output device 1140 can include a display unit for displaying graphical user interfaces.

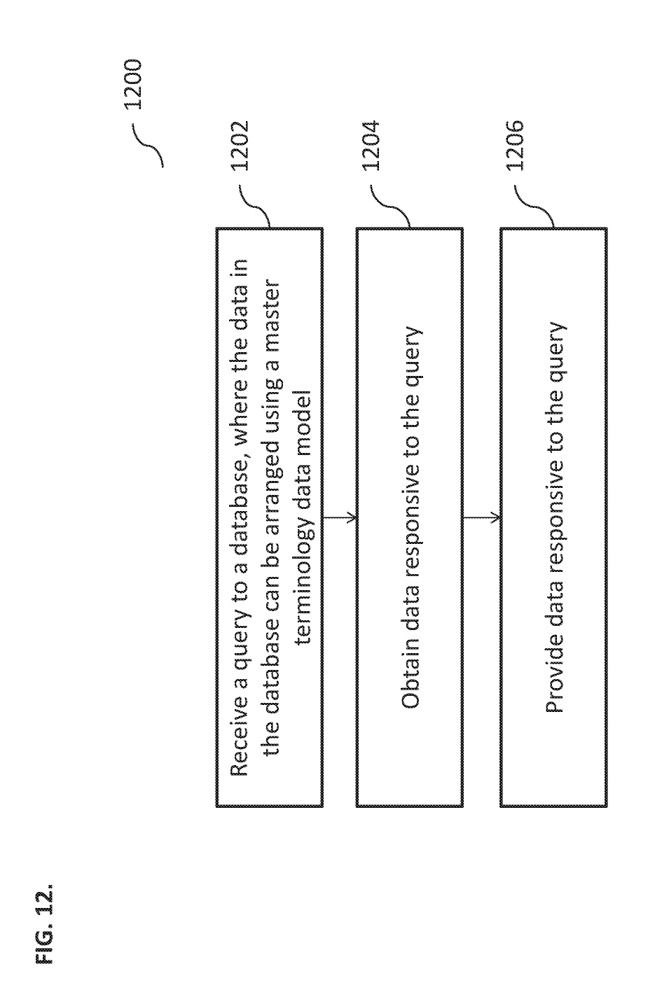

FIG. 12 illustrates an exemplary process 1200 for querying data, according to some implementations of the current subject matter. At 1202, a query to a database can be received. The query can include one or more parameters (e.g., search terms). Data in the database can be arranged using a master terminology data model, where the master terminology data model can contain a mapping of one or more terminology structures. At 1204, data responsive to the query can be obtained based on at least one parameter of the query. The data can be obtained by traversing the database in accordance with the mapping. The parameter can be an element of a first terminology structure in the plurality of terminology structures. The traversing can include at least one of the following. Based on the parameter, at least one site element contained in a second terminology structure in the plurality of terminology structures can be determined. At least one site element can identify data in the database for inclusion in the data responsive to the query. Additionally, at least one referenced element contained in the second terminology structure can be determined based on the parameter. The referenced element can identify data in the database being related to the data responsive to the query. At 1206, data responsive to the query can be provided in accordance with at least one of: the determined site element and the determined referenced element.

In some implementations, the structured master terminology data model can use a mapping of terms in two or more terminology structures and/or coding systems, e.g., ICD-10 and ICD-O. The structured data model can be a new terminology structure (e.g., cancer terminology), where the terminology can include a plurality of levels (level 0: “Tumor Registry” (e.g., top level), level 1: tumor site (or any other aspect of the cancer), etc.). Data can be mapped and structured using various aspects of the oncology data (e.g., tumor site, morphology (histology and behavior), tumor grade, tumor stage, cancer-specific factors, treatment, recurrence, multiple primary diagnoses, etc.). Further, specific data can be mapped between existing terminology structures using specific aspects of the cancer (e.g., diagnoses) to provide additional oncology data in the master terminology for assisting user in building/running of queries. In some implementations, synonyms in the oncology terminology can be used to allow the user to search for more colloquial terms for ease of use and for the purposes of creating the master terminology data model. In some implementations, a provider map to represent oncology data (e.g., tumor morphology, site-to-morphology, oncology qualifiers, etc.) can be generated so that the data can be appropriately loaded in accordance with the master terminology for querying purposes. In some implementations, the queries can be generated in free form/text and then translated into appropriate parameters based on the master terminology, where the resulting data can be presented via a user interface and/or in any other fashion. The queries can also be built using specific codes of the master terminology.

In some implementations, the current subject matter can include one or more of the following optional features. The first terminology structure can include terminology from International Classification of Disease (ICD-10) and the second terminology structure can include terminology from International Classification of Disease-Oncology (ICD-O). At least one site element can identify at least one of the following: a site of a tumor in a body of a patient, a tumor type, a biomarker, a mutation, a genomic biomarker, a genomic biomarker mutation, and any combination thereof. At least one referenced element can be determined based on the at least one site element. At least one referenced element can include at least one of the following: a tumor stage, a tumor grade, at least one cancer specific factor, at least one treatment, a tumor recurrence, at least one multiple primary diagnosis, morphology, and any combination thereof. Morphology can be determined based on the second terminology structure.

In some implementations, data can be obtained by selecting, based on the morphology, data responsive to the query.

In some implementations, at least one referenced element can include at least one of the following: a tumor stage, a tumor grade, at least one cancer specific factor, at least one treatment, a tumor recurrence, at least one multiple primary diagnosis, and any combination thereof. At least one site element can contain a morphology determined based on the parameter using the first terminology structure. Data in the database corresponding to the morphology can be included in the data responsive to the query.

The foregoing is considered as illustrative only of the principles of the invention. Further, since numerous modifications and changes will readily occur to those skilled in the art, it is not described to limit the invention to the exact construction and operation shown and described and accordingly, all suitable modifications and equivalents may be resorted to, falling within the scope of the invention.

Having described illustrative embodiments of the current subject matter with reference to the accompanying drawings, it will be appreciated that the current subject matter is not limited to the illustrated embodiments and that various changes and modifications can be effected therein by one of ordinary skill in the art without departing from the scope or spirit of the current subject matter as defined by the appended claims. Further modifications of the current subject matter can also occur to persons skilled in the art and all such are deemed to fall within the spirit and scope of the invention as defined by the appended claims.

Although particular embodiments have been disclosed herein in detail, this has been done by way of example and for purposes of illustration only, and is not intended to be limiting. In particular, it is contemplated by the inventors that various substitutions, alterations, and modifications may be made without departing from the spirit and scope of the disclosed embodiments. Other aspects, advantages, and modifications are considered to be within the scope of the disclosed and claimed embodiments, as well as other inventions disclosed herein. The claims presented hereafter are merely representative of some of the embodiments of the inventions disclosed herein. Other, presently unclaimed embodiments and inventions are also contemplated. The inventors reserve the right to pursue such embodiments and inventions in later claims and/or later applications claiming common priority.

As used herein, the term “user” can refer to any entity including a person or a computer or any other device.

Although ordinal numbers such as first, second, and the like can, in some situations, relate to an order; as used in this document ordinal numbers do not necessarily imply an order. For example, ordinal numbers can be merely used to distinguish one item from another. For example, to distinguish a first event from a second event, but need not imply any chronological ordering or a fixed reference system (such that a first event in one paragraph of the description can be different from a first event in another paragraph of the description).

To provide for interaction with a user, the subject matter described herein can be implemented on a computer having a display device, such as for example a cathode ray tube (CRT) or a liquid crystal display (LCD) monitor for displaying information to the user and a keyboard and a pointing device, such as for example a mouse or a trackball, by which the user can provide input to the computer. Other kinds of devices can be used to provide for interaction with a user as well. For example, feedback provided to the user can be any form of sensory feedback, such as for example visual feedback, auditory feedback, or tactile feedback; and input from the user can be received in any form, including, but not limited to, acoustic, speech, or tactile input.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations can be within the scope of the following claims.

### APPENDIX A

### Tumor Registry

### Ontology

The ontology used by the current subject matter is based on the North American Association of Central Cancer Registries (NAACCR, http://www.naaccr.org/).

The following is an analysis of the subset of Tumor Registry data based on the above ontology.

### Primary Cancer Diagnosis, Histology & Staging

Kind of cancer (typically anatomic location with exception of blood malignancies), type of tissue (histology) and stage are the mainstays of oncology data.

ICD-O is a standard vocabulary used to code the kind of cancer (also known as the topography code; specifies site) and type of tissue (also known as the behavior code; specifies tissue histology and aggressiveness of the tumor).

Below is a top-level list of kinds of cancer (organized primarily by body site):
BLOOD, BONE MARROW, HEMATOPOIETIC AND RETICULOENDOTHELIAL SYSTEM C42
BONES, JOINTS AND ARTICULAR CARTILAGE OF LIMBS C40-C41
BRAIN AND OTHER PARTS OF CENTRAL NERVOUS SYSTEM C70-C72
BREAST C50
CONNECTIVE, SUBCUTANEOUS AND OTHER SOFT TISSUES C49
DIGESTIVE ORGANS C15-C26
ENDOCRINE GLANDS AND RELATED STRUCTURES C73-C75
EYE AND ADNEXA C69
FEMALE GENITAL ORGANS C51-C58
LIP, ORAL CAVITY AND PHARYNX C00-C14
LYMPH NODES C77
MALE GENITAL ORGANS C60-C63
OTHER AND ILL-DEFINED SITES C76
PERIPHERAL NERVES AND AUTONOMIC NERVOUS SYSTEM C47
RESPIRATORY SYSTEM AND INTRATHORACIC ORGANS C30-C39
RETROPERITONEUM AND PERITONEUM C48
SKIN C44
URINARY ORGANS C64-C68

Note that these codes (letter C followed by 2 or 3 digits) represent only malignant neoplasms. Benign, in-situ or uncertain/unknown neoplasms (ICD-O codes starting with letter D) are not included in this ontology.

For every cancer kind, the Tumor Registry captures tissue histology and tumor stage. This ontology, designed for i2b2 before it was able to support multiple modifiers per fact, modeled histology and staging as children of each kind of cancer. In other words, the ICD-O-based hierarchy of body sites (see above) was “interrupted” at the level of last parent node before terminal nodes. At this level, two additional child nodes were inserted in every sub-tree: histology and stage. Here is an example of how this looks for colon and pancreatic cancer (histology and stage additions in red):
DIGESTIVE ORGANS C15-C26
   ANUS AND ANAL CANAL C21
   COLON C18
      Appendix C181
      Ascending colon C182
      Cecum C180
      Colon, NOS C189
      Descending colon C186
      Hepatic flexure of colon C183
      Overlapping lesion of colon C188
      Sigmoid colon C187
      Splenic flexure of colon C185
      Transverse colon C184
      Histology
      Stage, Grade, Behavior
   ESOPHAGUS C15
   GALLBLADDER C23
   LIVER AND INTRAHEPATIC BILE DUCTS C22
   OTHER AND ILL-DEFINED DIGESTIVE ORGANS C26
   OTHER AND UNSPECIFIED PARTS OF BILIARY TRACT C24
   PANCREAS C25
      Body of pancreas C251
      Head of pancreas C250
      Islets of Langerhans C254
      Other specified parts of pancreas C257
      Overlapping lesion of pancreas C258
      Pancreas, NOS C259
      Pancreatic duct C253
      Tail of pancreas C252
      Histology
      Stage, Grade, Behavior
   RECTOSIGMOID JUNCTION C19
   RECTUM C20
   SMALL INTESTINE C17
   STOMACH C16

The approach of inserting histology and staging “folders” as children into every sub-tree of ICD-O hierarchy works well in i2b2 web client where primary mode of interaction with the ontology is by browsing the set of nested folders.

### Additional Data

The last parent node (the parent of terminal nodes) in ICD-O hierarchy of kinds of cancer is associated with a number of i2b2 modifiers:
Age at diagnosis—based on value in years
Date of diagnosis—[no pop-up]
Primary Tumor Sequence—[no pop-up]
Survival (months from date of DX)—based on value in months
Survival disease-free (months from date of DX)—based on value in months
Year of 1st contact at the institution—based on 4-digit year

Some of these, such as age at and date of diagnoses as well as survival appear to be very important for oncology-related cohort identification.

### Histology

Each Histology folder contains a list of histologies that are possible for a given kind of cancer. These are also coded to ICD-O vocabulary for histology and tumor behavior.

Staging Each Stage folder contains a list of stages that are specific to a given kind of cancer. A tumor's stage is determined using 3 parameters: tumor size (T), number of lymph nodes involved (N), and presence or absence of metastasis (M). The system is frequently referred to as the TNM Stage. Jack's ontology captures raw values for TNM, both Clinical (typically based on imaging studies) and Pathological (based on tissue examination). T, N and M are represented as individual concepts with enumerated modifiers for possible values of T, N, and M for every particular kind of cancer.

Stage is represented as 3 concepts: best, clinical and pathological. Each is associated with an enumerated modifier with possible values for this cancer's stage (for example, Stage 1, Stage 1A, Stage 2, etc.).

Ontology contains two additional concepts in Stage folder: grade and behavior. Each is a concept associated with an enumerated modifier. Grade has values such as well differentiated, poorly differentiated, anaplastic, etc. Behavior has values such as benign, malignant, in situ, etc. Note that behavior is usually represented as a single digit addition to the 4-digit ICD-O histology code and separated from it by a “/”

### CS Site Specific Factors

Collaborative Stage (CS) Specific Factors are sets of cancer-specific data elements. The ontology limits these to the following sites only:
BREAST
COLON
COLON—GIST
COLON—NET
LUNG
PLEURA
PANCREAS
PROSTATE

The data is highly specific to a given cancer and will be extremely valuable for cohort identification. For example, breast cancer specific factors include ER/PR/HER2neu status and prostate cancer specific factors include Gleason scores.

### Treatment

The following top level treatment modalities are available in the ontology:
Chemotherapy
Diagnostic (ex, biopsy)
Endocrine Treatment
Hormone therapy
Immunotherapy
Other treatment
Palliative
Radiation
Surgery
Transplant Procedure

Some of these have child nodes. For example, “Chemotherapy, multiple agents (combination regimen)” and “Chemotherapy, single agent” are found under Chemotherapy.

### Recurrence

Recurrence documents first recurrence of the tumor either locally, regionally or at a distant site. There is also a modifier “Months from initial Dx to 1st Recurrence” with values in months.

This information may not be highly valuable for cohort identification.

### Multiple Primary Diagnoses

The following facts are available regarding multiple primaries:
Multiple malignant primaries
Multiple non-malignant primaries
Single malignant primary only (no multiple)
Single non-malignant primary only (no multiple)

### APPENDIX B

Scenario 1: ICD-10 Diagnosis mapped to ICD-O Site only

User selects ICD-10:D48 “Neoplasm of uncertain behavior of other and unspecified sites”

### Mapping for ICD-10:D48

Column “Include . . . ” is from ICD-10 to ICD-O mapping. Column “Referenced . . . ” is pre-generated by (1) taking “include” mapping to site, (2) traversing children of ICD-10 code to take their “include” mappings to site, (3) stripping significant digit to get to top-level ICD-O site code, (4) taking distinct superset of #3.

| | | Include | |
|---|---|---|---|
| | | ICD-O | Referenced |
| ICD-10 | STR | Site | ICD-O Site |
| D48 | Neoplasm of uncertain behavior of other and unspecified sites | C76 | C76, C41, |
| | | | C49, C47, |
| | | | C48, C44, |
| | | | C50 |
| D48.0 | Neoplasm of uncertain behavior of bone and articular cartilage | C41 | C41 |
| D48.1 | Neoplasm of uncertain behavior of connective and other soft tissue | C49 | C49 |
| D48.2 | Neoplasm of uncertain behavior of peripheral nerves and autonomic | C47 | C47 |
| | nervous system | | |
| D48.3 | Neoplasm of uncertain behavior of retroperitoneum | C48.0 | C48 |
| D48.4 | Neoplasm of uncertain behavior of peritoneum | C48.2 | C48 |
| D48.5 | Neoplasm of uncertain behavior of skin | C44 | C44 |
| D48.6 | Neoplasm of uncertain behavior of breast | C50 | C50 |
| D48.60 | Neoplasm of uncertain behavior of unspecified breast | C50 | C50 |
| D48.61 | Neoplasm of uncertain behavior of right breast | C50 | C50 |
| D48.62 | Neoplasm of uncertain behavior of left breast | C50 | C50 |
| D48.7 | Neoplasm of uncertain behavior of other specified sites | C76 | C76 |
| D48.9 | Neoplasm of uncertain behavior, unspecified | C76 | C76 |

### Site to Morphologies

These morphologies are presented to the user in oncology pop-up and are available for selection. Filled with the unique set of every morphology for every “referenced site,” derived from morphology-to-site relationships from the Master Terminology and augmented by provider data. When generating the query, we may generate combinations that do not apply but the result should be a no-op.

| ICD-O | | |
|---|---|---|
| Site | Description | Morphologies |
| C41 | BONES, JOINTS AND ARTICULAR | 9330/0, 9330/3, |
| | CARTILAGE OF OTHER AND | 9290/0, 9290/3, |
| | UNSPECIFIED SITES | etc. |
| C44 | SKIN | 8211/3, 8211/0, |
| | | 8573/3, etc. |
| C47 | PERIPHERAL NERVES AND AUTONOMIC | . . . |
| | NERVOUS SYSTEM | |
| C48 | RETROPERITONEUM AND PERITONEUM | |
| C49 | CONNECTIVE, SUBCUTANEOUS AND | |
| | OTHER SOFT TISSUES | |
| C50 | BREAST | |
| C76 | Neoplasm of uncertain behavior of skin | |

### Example—User Selects

ICD-10:D48
Stage 1
Morphology 9330/3

Note that Tumor Registry data for primary site is represented as ICD-O site code (e.g., TR:C48.2).

### Query:

ICD-10:D48 OR ICD-10:D48.0 OR ICD-10:D48.1 OR ICD-10:D48.2 OR ICD-10:D48.3 OR ICD-10:D48.4 OR ICD-10:D48.5 OR ICD-10:D48.6 OR ICD-10:D48.60 OR ICD-10:D48.61 OR ICD-10:D48.62 OR ICD-10:D48.7 OR ICD-10:D48.9 OR TR:C76 OR TR:C41 OR TR:C49 OR TR:C47 OR TR:C48.0 OR TR:C48.2 OR TR:C44 OR TR:C50
AND TR:C41|S1 OR TR:C491|S1 OR TR:C47|S1 OR TR:C48|S1 OR TR:C44|S1 OR TR:C50|S1 OR TR:C761|S1
AND TR:C41|9330/3 OR TR:C49|9330/3 OR TR:C47|9330/3 OR TR:C48|9330/3 OR TR:C44|9330/3 OR TR:C50|9330/3 OR TR:C76|9330/3

### Scenario 2: ICD-10 Diagnosis Mapped Primarily to Site and Secondarily to Morphology

User selects ICD-10:C44.31 “Basal cell carcinoma of skin of other and unspecified parts of face”

### Mapping for ICD-10:C44.31

| | | Include | Referenced | Include | |
|---|---|---|---|---|---|
| | | ICD-O | ICD-O | ICD-O | |
| ICD-10 | STR | Site | Site | Morphology | Primary |
| C44.31 | Basal cell carcinoma of skin | C44.3 | C44 | 8090/3 | S |
| | of other and unspecified parts | | | | |
| | of face | | | | |
| C44.310 | Basal cell carcinoma of skin | C44.3 | C44 | 8090/3 | S |
| | of unspecified parts of face | | | | |
| C44.311 | Basal cell carcinoma of skin | C44.3 | C44 | 8090/3 | S |
| | of nose | | | | |
| C44.319 | Basal cell carcinoma of skin | C44.3 | C44 | 8090/3 | S |
| | of other parts of face | | | | |

### Site to Morphologies

User is not able to select morphologies in this scenario since morphology is pre-defined in ICD-10 to ICD-O mapping. List of morphologies is pre-generated by (1) taking “include” mapping to morphology, (2) traversing children of ICD-10 code to take their “include” morphology mappings, and (3) taking distinct superset of ##1-2. Here all children of ICD-10:44.31 are mapped to the same morphology ICD-O: 8090/3.

### Example—User Selects

ICD-10:C44.31
Stage 2

Tumor Registry data represents primary site as TR:C44.3 and morphology as TR:C4418090/3. Note that ICD-O site preceding ICD-O morphology code is a top-level site (i.e., significant digit is stripped).

### Query to Contain:

ICD-10:C44.31 OR ICD-10:C44.310 OR ICD-10:C44.311 OR ICD-10:C44.319 OR (TR:C44.3 AND TR:C44|8090/3)
AND TR:C44|S2

This extends the query logic. It accommodates finding patients where a site and morphology are defined by the ICD-10 term but may exist in one or both areas

Note that no histology list is displayed in oncology pop-up in this scenario since morphology is pre-defined in the mapping

### Scenario 3: ICD-10 Diagnosis Mapped to Morphology Only

User selects ICD-10:C81 “Hodgkin lymphoma”

### Mapping for ICD-10:C81

ICD-10:C81 is mapped to morphology (ICD-O:9650/3) and has no ICD-O site mappings. Column “Include ICD-O Morphology” is pre-generated by (1) taking mapped morphology code, (2) traversing children of that ICD-10 code and adding morphology codes for children, if any, and (3) taking a distinct superset of ##1-2.

Referenced ICD-O sites are pre-generated by (1) traversing the children of ICD-10:C81 (get C77.* and C42.2) and deriving top-level ICD-O sites by stripping the significant digit if applicable (get C77, C42), (2) deriving a list of sites from “included” morphologies via the morphology-to-site relationships (C77, C42, C37, C16), (3) augmenting that with provider data (C77, C80, C07, C34, C42, C41, C38, C16), and (4) taking a distinct superset of the above sites.

| | | Include | | Mapped | |
|---|---|---|---|---|---|
| | | ICD-O | Referenced | ICD-O | Include |
| ICD-10 | STR | Site | ICD-O Site | Morphology | ICD-O Morphology |
| C81 | Hodgkin lymphoma | | C77, C42, | 9650/3 | 9650/3, 9659/3, |
| | | | C37, C16, | | 9663/3, 9652/3, |
| | | | C80, C07, | | 9653/3, 9651/3 |
| | | | C34, C41, C38 | | |
| C81.0 | Nodular lymphocyte | | C77, C42, | 9659/3 | 9659/3 |
| | predominant Hodgkin | | C37, C16, | | |
| | lymphoma | | C80, C07, | | |
| | | | C34, C41, C38 | | |
| C81.00 | Nodular lymphocyte | | C77, C42, | 9659/3 | 9659/3 |
| | predominant Hodgkin | | C37, C16, | | |
| | lymphoma, unspecified | | C80, C07, | | |
| | site | | C34, C41, C38 | | |
| C81.01 | Nodular lymphocyte | C77.0 | C77 | 9659/3 | 9659/3 |
| | predominant Hodgkin | | | | |
| | lymphoma, lymph nodes | | | | |
| | of head, face, and neck | | | | |
| C81.02 | Nodular lymphocyte | C77.1 | C77 | 9659/3 | 9659/3 |
| | predominant Hodgkin | | | | |
| | lymphoma, intrathoracic | | | | |
| | lymph nodes | | | | |
| C81.03 | Nodular lymphocyte | C77.2 | C77 | 9659/3 | 9659/3 |
| | predominant Hodgkin | | | | |
| | lymphoma, intra- | | | | |
| | abdominal lymph nodes | | | | |
| C81.04 | Nodular lymphocyte | C77.3 | C77 | 9659/3 | 9659/3 |
| | predominant Hodgkin | | | | |
| | lymphoma, lymph nodes | | | | |
| | of axilla and upper limb | | | | |
| C81.05 | Nodular lymphocyte | C77.4 | C77 | 9659/3 | 9659/3 |
| | predominant Hodgkin | | | | |
| | lymphoma, lymph nodes | | | | |
| | of inguinal region and | | | | |
| | lower limb | | | | |
| C81.06 | Nodular lymphocyte | C77.5 | C77 | 9659/3 | 9659/3 |
| | predominant Hodgkin | | | | |
| | lymphoma, intrapelvic | | | | |
| | lymph nodes | | | | |
| C81.07 | Nodular lymphocyte | C42.2 | C44 | 9659/3 | 9659/3 |
| | predominant Hodgkin | | | | |
| | lymphoma, spleen | | | | |
| C81.08 | Nodular lymphocyte | C77.8 | C77 | 9659/3 | 9659/3 |
| | predominant Hodgkin | | | | |
| | lymphoma, lymph nodes | | | | |
| | of multiple sites | | | | |
| C81.09 | Nodular lymphocyte | | C77, C42, | 9659/3 | 9659/3 |
| | predominant Hodgkin | | C37, C16, | | |
| | lymphoma, extranodal and | | C80, C07, | | |
| | solid organ sites | | C34, C41, C38 | | |
| C81.1 | Nodular sclerosis classical | | C77, C42, | 9663/3 | 9663/3 |
| | Hodgkin lymphoma | | C37, C16, | | |
| | | | C80, C07, | | |
| | | | C34, C41, C38 | | |
| C81.10 | Nodular sclerosis classical | | C77, C42, | 9663/3 | 9663/3 |
| | Hodgkin lymphoma, | | C37, C16, | | |
| | unspecified site | | C80, C07, | | |
| | | | C34, C41, C38 | | |
| C81.11 | Nodular sclerosis classical | C77.0 | C77 | 9663/3 | 9663/3 |
| | Hodgkin lymphoma, | | | | |
| | lymph nodes of head, | | | | |
| | face, and neck | | | | |
| C81.12 | Nodular sclerosis classical | C77.1 | C77 | 9663/3 | 9663/3 |
| | Hodgkin lymphoma, | | | | |
| | intrathoracic lymph nodes | | | | |
| C81.13 | Nodular sclerosis classical | C77.2 | C77 | 9663/3 | 9663/3 |
| | Hodgkin lymphoma, intra- | | | | |
| | abdominal lymph nodes | | | | |
| C81.14 | Nodular sclerosis classical | C77.3 | C77 | 9663/3 | 9663/3 |
| | Hodgkin lymphoma, | | | | |
| | lymph nodes of axilla and | | | | |
| | upper limb | | | | |
| C81.15 | Nodular sclerosis classical | C77.4 | C77 | 9663/3 | 9663/3 |
| | Hodgkin lymphoma, | | | | |
| | lymph nodes of inguinal | | | | |
| | region and lower limb | | | | |
| C81.16 | Nodular sclerosis classical | C77.5 | C77 | 9663/3 | 9663/3 |
| | Hodgkin lymphoma, | | | | |
| | intrapelvic lymph nodes | | | | |
| C81.17 | Nodular sclerosis classical | C42.2 | C44 | 9663/3 | 9663/3 |
| | Hodgkin lymphoma, | | | | |
| | spleen | | | | |
| C81.18 | Nodular sclerosis classical | C77.8 | C77 | 9663/3 | 9663/3 |
| | Hodgkin lymphoma, | | | | |
| | lymph nodes of multiple | | | | |
| | sites | | | | |
| C81.19 | Nodular sclerosis classical | | C77, C42, | 9663/3 | 9663/3 |
| | Hodgkin lymphoma, | | C37, C16, | | |
| | extranodal and solid organ | | C80, C07, | | |
| | sites | | C34, C41, C38 | | |
| C81.2 | Mixed cellularity classical | | C77, C42, | 9652/3 | 9652/3 |
| | Hodgkin lymphoma | | C37, C16, | | |
| | | | C80, C07, | | |
| | | | C34, C41, C38 | | |
| C81.20 | Mixed cellularity classical | | C77, C42, | 9652/3 | 9652/3 |
| | Hodgkin lymphoma, | | C37, C16, | | |
| | unspecified site | | C80, C07, | | |
| | | | C34, C41, C38 | | |
| C81.21 | Mixed cellularity classical | C77.0 | C77 | 9652/3 | 9652/3 |
| | Hodgkin lymphoma, | | | | |
| | lymph nodes of head, | | | | |
| | face, and neck | | | | |
| C81.22 | Mixed cellularity classical | C77.1 | C77 | 9652/3 | 9652/3 |
| | Hodgkin lymphoma, | | | | |
| | intrathoracic lymph nodes | | | | |
| C81.23 | Mixed cellularity classical | C77.2 | C77 | 9652/3 | 9652/3 |
| | Hodgkin lymphoma, intra- | | | | |
| | abdominal lymph nodes | | | | |
| C81.24 | Mixed cellularity classical | C77.3 | C77 | 9652/3 | 9652/3 |
| | Hodgkin lymphoma, | | | | |
| | lymph nodes of axilla and | | | | |
| | upper limb | | | | |
| C81.25 | Mixed cellularity classical | C77.4 | C77 | 9652/3 | 9652/3 |
| | Hodgkin lymphoma, | | | | |
| | lymph nodes of inguinal | | | | |
| | region and lower limb | | | | |
| C81.26 | Mixed cellularity classical | C77.5 | C77 | 9652/3 | 9652/3 |
| | Hodgkin lymphoma, | | | | |
| | intrapelvic lymph nodes | | | | |
| C81.27 | Mixed cellularity classical | C42.2 | C44 | 9652/3 | 9652/3 |
| | Hodgkin lymphoma, | | | | |
| | spleen | | | | |
| C81.28 | Mixed cellularity classical | C77.8 | C77 | 9652/3 | 9652/3 |
| | Hodgkin lymphoma, | | | | |
| | lymph nodes of multiple | | | | |
| | sites | | | | |
| C81.29 | Mixed cellularity classical | | C77, C42, | 9652/3 | 9652/3 |
| | Hodgkin lymphoma, | | C37, C16, | | |
| | extranodal and solid organ | | C80, C07, | | |
| | sites | | C34, C41, C38 | | |
| C81.3 | Lymphocyte depleted | | C77, C42, | 9653/3 | 9653/3 |
| | classical Hodgkin | | C37, C16, | | |
| | lymphoma | | C80, C07, | | |
| | | | C34, C41, C38 | | |
| C81.30 | Lymphocyte depleted | | C77, C42, | 9653/3 | 9653/3 |
| | classical Hodgkin | | C37, C16, | | |
| | lymphoma, unspecified | | C80, C07, | | |
| | site | | C34, C41, C38 | | |
| C81.31 | Lymphocyte depleted | C77.0 | C77 | 9653/3 | 9653/3 |
| | classical Hodgkin | | | | |
| | lymphoma, lymph nodes | | | | |
| | of head, face, and neck | | | | |
| C81.32 | Lymphocyte depleted | C77.1 | C77 | 9653/3 | 9653/3 |
| | classical Hodgkin | | | | |
| | lymphoma, intrathoracic | | | | |
| | lymph nodes | | | | |
| C81.33 | Lymphocyte depleted | C77.2 | C77 | 9653/3 | 9653/3 |
| | classical Hodgkin | | | | |
| | mphoma, intra- | | | | |
| | abdominal lymph nodes | | | | |
| C81.34 | Lymphocyte depleted | C77.3 | C77 | 9653/3 | 9653/3 |
| | classical Hodgkin | | | | |
| | lymphoma, lymph nodes | | | | |
| | of axilla and upper limb | | | | |
| C81.35 | Lymphocyte depleted | C77.4 | C77 | 9653/3 | 9653/3 |
| | classical Hodgkin | | | | |
| | lymphoma, lymph nodes | | | | |
| | of inguinal region and | | | | |
| | lower limb | | | | |
| C81.36 | Lymphocyte depleted | C77.5 | C77 | 9653/3 | 9653/3 |
| | classical Hodgkin | | | | |
| | lymphoma, intrapelvic | | | | |
| | lymph nodes | | | | |
| C81.37 | Lymphocyte depleted | C42.2 | C44 | 9653/3 | 9653/3 |
| | classical Hodgkin | | | | |
| | lymphoma, spleen | | | | |
| C81.38 | Lymphocyte depleted | C77.8 | C77 | 9653/3 | 9653/3 |
| | classical Hodgkin | | | | |
| | lymphoma, lymph nodes | | | | |
| | of multiple sites | | | | |
| C81.39 | Lymphocyte depleted | | C77, C42, | 9653/3 | 9653/3 |
| | classical Hodgkin | | C37, C16, | | |
| | lymphoma, extranodal and | | C80, C07, | | |
| | solid organ sites | | C34, C41, C38 | | |
| C81.4 | Lymphocyte-rich classical | | C77, C42, | 9651/3 | 9651/3 |
| | Hodgkin lymphoma | | C37, C16, | | |
| | | | C80, C07, | | |
| | | | C34, C41, C38 | | |
| C81.40 | Lymphocyte-rich classical | | C77, C42, | 9651/3 | 9651/3 |
| | Hodgkin lymphoma, | | C37, C16, | | |
| | unspecified site | | C80, C07, | | |
| | | | C34, C41, C38 | | |
| C81.41 | Lymphocyte-rich classical | C77.0 | C77 | 9651/3 | 9651/3 |
| | Hodgkin lymphoma, | | | | |
| | lymph nodes of head, | | | | |
| | face, and neck | | | | |
| C81.42 | Lymphocyte-rich classical | C77.1 | C77 | 9651/3 | 9651/3 |
| | Hodgkin lymphoma, | | | | |
| | intrathoracic lymph nodes | | | | |
| C81.43 | Lymphocyte-rich classical | C77.2 | C77 | 9651/3 | 9651/3 |
| | Hodgkin lymphoma, intra- | | | | |
| | abdominal lymph nodes | | | | |
| C81.44 | Lymphocyte-rich classical | C77.3 | C77 | 9651/3 | 9651/3 |
| | Hodgkin lymphoma, | | | | |
| | lymph nodes of axilla and | | | | |
| | upper limb | | | | |
| C81.45 | Lymphocyte-rich classical | C77.4 | C77 | 9651/3 | 9651/3 |
| | Hodgkin lymphoma, | | | | |
| | lymph nodes of inguinal | | | | |
| | region and lower limb | | | | |
| C81.46 | Lymphocyte-rich classical | C77.5 | C77 | 9651/3 | 9651/3 |
| | Hodgkin lymphoma, | | | | |
| | intrapelvic lymph nodes | | | | |
| C81.47 | Lymphocyte-rich classical | C42.2 | C44 | 9651/3 | 9651/3 |
| | Hodgkin lymphoma, | | | | |
| | spleen | | | | |
| C81.48 | Lymphocyte-rich classical | C77.8 | C77 | 9651/3 | 9651/3 |
| | Hodgkin lymphoma, | | | | |
| | lymph nodes of multiple | | | | |
| | sites | | | | |
| C81.49 | Lymphocyte-rich classical | | C77, C42, | 9651/3 | 9651/3 |
| | Hodgkin lymphoma, | | C37, C16, | | |
| | extranodal and solid organ | | C80, C07, | | |
| | sites | | C34, C41, C38 | | |
| C81.7 | Other classical Hodgkin | | C77, C42, | 9650/3 | 9650/3 |
| | lymphoma | | C37, C16, | | |
| | | | C80, C07, | | |
| | | | C34, C41, C38 | | |
| C81.70 | Other classical Hodgkin | | C77, C42, | 9650/3 | 9650/3 |
| | lymphoma, unspecified | | C37, C16, | | |
| | site | | C80, C07, | | |
| | | | C34, C41, C38 | | |
| C81.71 | Other classical Hodgkin | C77.0 | C77 | 9650/3 | 9650/3 |
| | lymphoma, lymph nodes | | | | |
| | of head, face, and neck | | | | |
| C81.72 | Other classical Hodgkin | C77.1 | C77 | 9650/3 | 9650/3 |
| | lymphoma, intrathoracic | | | | |
| | lymph nodes | | | | |
| C81.73 | Other classical Hodgkin | C77.2 | C77 | 9650/3 | 9650/3 |
| | lymphoma, intra- | | | | |
| | abdominal lymph nodes | | | | |
| C81.74 | Other classical Hodgkin | C77.3 | C77 | 9650/3 | 9650/3 |
| | lymphoma, lymph nodes | | | | |
| | of axilla and upper limb | | | | |
| C81.75 | Other classical Hodgkin | C77.4 | C77 | 9650/3 | 9650/3 |
| | lymphoma, lymph nodes | | | | |
| | of inguinal region and | | | | |
| | lower limb | | | | |
| C81.76 | Other classical Hodgkin | C77.5 | C77 | 9650/3 | 9650/3 |
| | lymphoma, intrapelvic | | | | |
| | lymph nodes | | | | |
| C81.77 | Other classical Hodgkin | C42.2 | C44 | 9650/3 | 9650/3 |
| | lymphoma, spleen | | | | |
| C81.78 | Other classical Hodgkin | C77.8 | C77 | 9650/3 | 9650/3 |
| | lymphoma, lymph nodes | | | | |
| | of multiple sites | | | | |
| C81.79 | Other classical Hodgkin | | C77, C42, | 9650/3 | 9650/3 |
| | lymphoma, extranodal and | | C37, C16, | | |
| | solid organ sites | | C80, C07, | | |
| | | | C34, C41, C38 | | |
| C81.9 | Hodgkin lymphoma, | | C77, C42, | 9650/3 | 9650/3 |
| | unspecified | | C37, C16, | | |
| | | | C80, C07, | | |
| | | | C34, C41, C38 | | |
| C81.90 | Hodgkin lymphoma, | | C77, C42, | 9650/3 | 9650/3 |
| | unspecified, unspecified | | C37, C16, | | |
| | site | | C80, C07, | | |
| | | | C34, C41, C38 | | |
| C81.91 | Hodgkin lymphoma, | C77.0 | C77 | 9650/3 | 9650/3 |
| | unspecified, lymph nodes | | | | |
| | of head, face, and neck | | | | |
| C81.92 | Hodgkin lymphoma, | C77.1 | C77 | 9650/3 | 9650/3 |
| | unspecified, intrathoracic | | | | |
| | lymph nodes | | | | |
| C81.93 | Hodgkin lymphoma, | C77.2 | C77 | 9650/3 | 9650/3 |
| | unspecified, intra- | | | | |
| | abdominal lymph nodes | | | | |
| C81.94 | Hodgkin lymphoma, | C77.3 | C77 | 9650/3 | 9650/3 |
| | unspecified, lymph nodes | | | | |
| | of axilla and upper limb | | | | |
| C81.95 | Hodgkin lymphoma, | C77.4 | C77 | 9650/3 | 9650/3 |
| | unspecified, lymph nodes | | | | |
| | of inguinal region and | | | | |
| | lower limb | | | | |
| C81.96 | Hodgkin lymphoma, | C77.5 | C77 | 9650/3 | 9650/3 |
| | unspecified, intrapelvic | | | | |
| | lymph nodes | | | | |
| C81.97 | Hodgkin lymphoma, | C42.2 | C44 | 9650/3 | 9650/3 |
| | unspecified, spleen | | | | |
| C81.98 | Hodgkin lymphoma, | C77.8 | C77 | 9650/3 | 9650/3 |
| | unspecified, lymph nodes | | | | |
| | of multiple sites | | | | |
| C81.99 | Hodgkin lymphoma, | | C77, C42, | 9650/3 | 9650/3 |
| | unspecified, extranodal | | C37, C16, | | |
| | and solid organ sites | | C80, C07, | | |
| | | | C34, C41, C38 | | |

### Site to Morphologies

The user is not able to select morphologies in this scenario since the ICD-10 term of interest has children with explicit mappings to morphologies. All permutations of these ICD-O morphologies with the list of “referenced” ICD-O sites will represent the full list of “included” morphologies. This list should be pre-generated and stored in Master Terminology.

### Example—User Selects

ICD-10:C81
Stage 3

### Query:

ICD-10:C81 OR ICD-10:C81.0 OR ICD-10:C81.00 OR ICD-10:C81.01 OR ICD-10:C81.02 OR ICD-10:C81.03 OR ICD-10:C81.04 OR ICD-10:C81.05 OR ICD-10:C81.06 OR ICD-10:C81.07 OR ICD-10:C81.08 OR ICD-10:C81.09 OR ICD-10:C81.1 OR ICD-10:C81.10 OR ICD-10:C81.11 OR ICD-10:C81.12 OR ICD-10:C81.13 OR ICD-10:C81.14 OR ICD-10:C81.15 OR ICD-10:C81.16 OR ICD-10:C81.17 OR ICD-10:C81.18 OR ICD-10:C81.19 OR ICD-10:C81.2 OR ICD-10:C81.20 OR ICD-10:C81.21 OR ICD-10:C81.22 OR ICD-10:C81.23 OR ICD-10:C81.24 OR ICD-10:C81.25 OR ICD-10:C81.26 OR ICD-10:C81.27 OR ICD-10:C81.28 OR ICD-10:C81.29 OR ICD-10:C81.3 OR ICD-10:C81.30 OR ICD-10:C81.31 OR ICD-10:C81.32 OR ICD-10:C81.33 OR ICD-10:C81.34 OR ICD-10:C81.35 OR ICD-10:C81.36 OR ICD-10:C81.37 OR ICD-10:C81.38 OR ICD-10:C81.39 OR ICD-10:C81.4 OR ICD-10:C81.40 OR ICD-10:C81.41 OR ICD-10:C81.42 OR ICD-10:C81.43 OR ICD-10:C81.44 OR ICD-10:C81.45 OR ICD-10:C81.46 OR ICD-10:C81.47 OR ICD-10:C81.48 OR ICD-10:C81.49 OR ICD-10:C81.7 OR ICD-10:C81.70 OR ICD-10:C81.71 OR ICD-10:C81.72 OR ICD-10:C81.73 OR ICD-10:C81.74 OR ICD-10:C81.75 OR ICD-10:C81.76 OR ICD-10:C81.77 OR ICD-10:C81.78 OR ICD-10:C81.79 OR ICD-10:C81.9 OR ICD-10:C81.90 OR ICD-10:C81.91 OR ICD-10:C81.92 OR ICD-10:C81.93 OR ICD-10:C81.94 OR ICD-10:C81.95 OR ICD-10:C81.96 OR ICD-10:C81.97 OR ICD-10:C81.98 OR ICD-10:C81.99
OR TR:C77|9650/3 OR TR:C42|9650/3 OR TR:C37|9650/3 OR TR:C16|9650/3 OR TR:C80|9650/3 OR TR:C07|9650/3 OR TR:C34|9650/3 OR TR:C41|9650/3 OR TR:C38|9650/3
OR TR:C77|9659/3 OR TR:C42|9659/3 OR TR:C37|9659/3 OR TR:C16|9659/3 OR TR:C80|9659/3 OR TR:C07|9659/3 OR TR:C34|9659/3 OR TR:C41|9659/3 OR TR:C38|9659/3
OR TR:C77|9663/3 OR TR:C42|9663/3 OR TR:C37|9663/3 OR TR:C16|9663/3 OR TR:C80|9663/3 OR TR:C07|9663/3 OR TR:C34|9663/3 OR TR:C41|9663/3 OR TR:C38|9663/3
OR TR:C77|9652/3 OR TR:C42|9652/3 OR TR:C37|9652/3 OR TR:C16|9652/3 OR TR:C80|9652/3 OR TR:C07|9652/3 OR TR:C34|9652/3 OR TR:C41|9652/3 OR TR:C38|9652/3
OR TR:C77|9653/3 OR TR:C42|9653/3 OR TR:C37|9653/3 OR TR:C16|9653/3 OR TR:C80|9653/3 OR TR:C07|9653/3 OR TR:C34|9653/3 OR TR:C41|9653/3 OR TR:C38|9653/3
OR TR:C77|9651/3 OR TR:C429651/3 OR TR:C37|9651/3 OR TR:C16|9651/3 OR TR:C80|9651/3 OR TR:C07|9651/3 OR TR:C34|9651/3 OR TR:C41|9651/3 OR TR:C38|9651/3
AND TR:C77|S3 OR TR:C42|S3 OR TR:C37|S3 OR TR:C16|S3 OR TR:C80|S3 OR TR:C07/S3 OR TR:C34|S3 OR TR:C41|S3 OR TR:C38|S3

### Scenario 4: ICD-10 Diagnosis Mapped Primarily to Morphology and Secondarily to Site

User selects ICD-10:C82.52 “Diffuse follicle center lymphoma, intrathoracic lymph nodes”

### Mapping for ICD-10:C82.52

Based on ICD-10 to ICD-O mapping, “included” ICD-O morphology is ICD-O:9690/3, and ICD-10:C82.52 has no children, so this is the only “included” morphology. ICD-10:C82.52 is also mapped to ICD-O site C77.1 and as there are no children, this is the only site. Referenced site, therefore, is C77 (stripping significant digit).

| | | | | Include | |
|---|---|---|---|---|---|
| | | ICD-O | Referenced | ICD-O | |
| ICD-10 | STR | Site | ICD-O Site | Morphology | Primary |
| C82.52 | Diffuse follicle | C77.1 | C77 | 9690/3 | M |
| | center | | | | |
| | lymphoma, | | | | |
| | intrathoracic | | | | |
| | lymph | | | | |
| | nodes | | | | |

### Site to Morphology

The user is not able to select morphologies in this scenario since ICD-10:C82.52 is explicitly mapped to ICD-O morphology.

### Example—User Selects

ICD-10:C82.52
Stage 4

Tumor Registry data represents morphology as TR:C77|9690/3 and site as TR:C77.1. Note that ICD-O site preceding ICD-O morphology code is a top-level site (i.e., significant digit is striped).

### Query to Contain:

ICD-10:C82.52 OR (TR:C77|9690/3 AND TR:C77.1)
AND TR:C77|S4

This extends the query logic.

What is claimed:

1. A computer implemented method, comprising
receiving a query to a database, the data being stored in accordance with at least one data model, the at least one data model containing at least one data node storing data and being structured in accordance with at least one master terminology containing a mapping of a plurality of terminology structures;
obtaining, based on at least one parameter of the query, data from the database responsive to the query by traversing the database in accordance with the mapping, the at least one parameter being an element of a first terminology structure in the plurality of terminology structures, the traversing including at least one of the following:
determining, based on the at least one parameter, at least one site element contained in a second terminology structure in the plurality of terminology structures, the at least one site element identifying data in the database for inclusion in the data responsive to the query;
determining, based on the at least one parameter, at least one referenced element contained in the second terminology structure, the at least one referenced element identifying data in the database being related to the data responsive to the query;
and
providing the data responsive to the query in accordance with the at least one of: the at least one determined site element and the at least one determined referenced element.

2. The method according to claim 1, wherein the first terminology structure includes terminology from International Classification of Disease (ICD-10) and the second terminology structure includes terminology from International Classification of Disease-Oncology (ICD-O).

3. The method according to claim 2, wherein the at least one site element identifying at least one of the following: a site of a tumor in a body of a patient, a tumor type, a biomarker, a mutation, a genomic biomarker, a genomic biomarker mutation, and any combination thereof.

4. The method according to claim 3, wherein the at least one referenced element is determined based on the at least one site element.

5. The method according to claim 4, wherein the at least one referenced element including at least one of the following: a tumor stage, a tumor grade, at least one cancer specific factor, at least one treatment, a tumor recurrence, at least one multiple primary diagnosis, morphology, and any combination thereof.

6. The method according to claim 5, wherein the morphology is determined based on the second terminology structure.

7. The method according to claim 6, wherein the obtaining includes
selecting, based on the morphology, data responsive to the query.

8. The method according to claim 4, wherein the at least one referenced element including at least one of the following: a tumor stage, a tumor grade, at least one cancer specific factor, at least one treatment, a tumor recurrence, at least one multiple primary diagnosis, and any combination thereof.

9. The method according to claim 8, wherein the at least one site element containing a morphology determined based on the at least one parameter using the first terminology structure, wherein data in the database corresponding to the morphology is included in the data responsive to the query.

10. A system comprising:
at least one programmable processor; and
a machine-readable medium storing instructions that, when executed by the at least one programmable processor, cause the at least one programmable processor to perform operations comprising:
receiving a query to a database, the data being stored in accordance with at least one data model, the at least one data model containing at least one data node storing data and being structured in accordance with at least one master terminology containing a mapping of a plurality of terminology structures;
obtaining, based on at least one parameter of the query, data from the database responsive to the query by traversing the database in accordance with the mapping, the at least one parameter being an element of a first terminology structure in the plurality of terminology structures, the traversing including at least one of the following:
determining, based on the at least one parameter, at least one site element contained in a second terminology structure in the plurality of terminology structures, the at least one site element identifying data in the database for inclusion in the data responsive to the query;
determining, based on the at least one parameter, at least one referenced element contained in the second terminology structure, the at least one referenced element identifying data in the database being related to the data responsive to the query;
and
providing the data responsive to the query in accordance with the at least one of: the at least one determined site element and the at least one determined referenced element.

11. The system according to claim 12, wherein the first terminology structure includes terminology from International Classification of Disease (ICD-10) and the second terminology structure includes terminology from International Classification of Disease-Oncology (ICD-O).

12. The system according to claim 11, wherein the at least one site element identifying at least one of the following: a site of a tumor in a body of a patient, a tumor type, a biomarker, a mutation, a genomic biomarker, a genomic biomarker mutation, and any combination thereof.

13. The system according to claim 12, wherein the at least one referenced element is determined based on the at least one site element.

14. The system according to claim 13, wherein the at least one referenced element including at least one of the following: a tumor stage, a tumor grade, at least one cancer specific factor, at least one treatment, a tumor recurrence, at least one multiple primary diagnosis, morphology, and any combination thereof.

15. The system according to claim 14, wherein the morphology is determined based on the second terminology structure.

16. The system according to claim 15, wherein the obtaining includes
selecting, based on the morphology, data responsive to the query.

17. The system according to claim 13, wherein the at least one referenced element including at least one of the following: a tumor stage, a tumor grade, at least one cancer specific factor, at least one treatment, a tumor recurrence, at least one multiple primary diagnosis, and any combination thereof.

18. The system according to claim 17, wherein the at least one site element containing a morphology determined based on the at least one parameter using the first terminology structure, wherein data in the database corresponding to the morphology is included in the data responsive to the query.

19. A computer program product comprising a non-transitory machine-readable medium storing instructions that, when executed by at least one programmable processor, cause the at least one programmable processor to perform operations comprising:
receiving a query to a database, the data being stored in accordance with at least one data model, the at least one data model containing at least one data node storing data and being structured in accordance with at least one master terminology containing a mapping of a plurality of terminology structures;
obtaining, based on at least one parameter of the query, data from the database responsive to the query by traversing the database in accordance with the mapping, the at least one parameter being an element of a first terminology structure in the plurality of terminology structures, the traversing including at least one of the following:
determining, based on the at least one parameter, at least one site element contained in a second terminology structure in the plurality of terminology structures, the at least one site element identifying data in the database for inclusion in the data responsive to the query;
determining, based on the at least one parameter, at least one referenced element contained in the second terminology structure, the at least one referenced element identifying data in the database being related to the data responsive to the query;
and
providing the data responsive to the query in accordance with the at least one of: the at least one determined site element and the at least one determined referenced element.

20. The computer program product according to claim 19, wherein the first terminology structure includes terminology from International Classification of Disease (ICD-10) and the second terminology structure includes terminology from International Classification of Disease-Oncology (ICD-O).

21. The computer program product according to claim 20, wherein the at least one site element identifying at least one of the following: a site of a tumor in a body of a patient, a tumor type, a biomarker, a mutation, a genomic biomarker, a genomic biomarker mutation, and any combination thereof.

22. The computer program product according to claim 21, wherein the at least one referenced element is determined based on the at least one site element.

23. The computer program product according to claim 22, wherein the at least one referenced element including at least one of the following: a tumor stage, a tumor grade, at least one cancer specific factor, at least one treatment, a tumor recurrence, at least one multiple primary diagnosis, morphology, and any combination thereof.

24. The computer program product according to claim 23, wherein the morphology is determined based on the second terminology structure.

25. The computer program product according to claim 24, wherein the obtaining includes
selecting, based on the morphology, data responsive to the query.

26. The computer program product according to claim 22, wherein the at least one referenced element including at least one of the following: a tumor stage, a tumor grade, at least one cancer specific factor, at least one treatment, a tumor recurrence, at least one multiple primary diagnosis, and any combination thereof.

27. The computer program product according to claim 26, wherein the at least one site element containing a morphology determined based on the at least one parameter using the first terminology structure, wherein data in the database corresponding to the morphology is included in the data responsive to the query.
